(19) United States
(12) Patent Application Publication
(10) Pub. No.: US 2022/0115092 A1
(43) Pub. Date: Apr. 14, 2022

(54) MACHINE LEARNING QUANTIFICATION OF TARGET ORGANISMS USING NUCLEIC ACID AMPLIFICATION ASSAYS

(71) Applicant: 3M INNOVATIVE PROPERTIES COMPANY, St. Paul, MN (US)

(72) Inventors: Wilfredo Dominguez-Nunez, Minneapolis, MN (US); Raj Rajagopal, Woodbury, MN (US); Nicholas A. Asendorf, St. Paul, MN (US); Saber Taghvaeeyan, Maple Grove, MN (US)

(21) Appl. No.: 17/431,196

(22) PCT Filed: Jan. 27, 2020
(86) PCT No.: PCT/IB2020/050607

§ 371 Date: Aug. 16, 2021

Related U.S. Application Data
(60) Provisional application No. 62/809,199, filed on Feb. 22, 2019.
(60) Provisional application No. 62/850,136, filed on May 20, 2019.

(51) Int. Cl. G16B 40/20; G16B 20/00; C12Q 1/06

(52) U.S. Cl. CPC G16B 40/20; C12Q 1/06; G16B 20/00

(57) ABSTRACT

In some examples, a system for amplifying and quantifying a target organism present in a sample includes a detection device configured to amplify and detect a nucleic acid associated with the target organism. The detection device configured to receive a sample and to amplify nucleic acid in the sample over an amplification cycle. The detection device is configured to capture a data set including measurements of the nucleic acid collected during the amplification cycle. The system further includes a computing device configured to receive the data set and to apply a machine learning system to the data set. The machine learning system is trained to estimate a quantity of the target organism present in the sample based on the measurements in the data set.

### TECHNICAL FIELD

This disclosure relates to systems and methods for detecting target organisms, and, in particular, to systems and methods for estimating quantities of a target organism.

### BACKGROUND

Foodborne bacterial infections and diseases are an ongoing threat to public health. Regulatory agencies such as the United States Department of Agriculture's Food Safety and Inspection Service respond to this threat by promulgating pathogen-reduction performance standards for pathogens (e.g., Salmonella and Campylobacter) in food, feed, water and corresponding processing environments. Some such pathogen-reduction standards apply presence/absence criteria while others require quantitative information on the pathogen.

Food, feed and water producers use quantitative techniques to determine the quantity of microorganisms, such as bacterial pathogens, in food, feed (e.g., animal feed), water and corresponding processing environments. Such producers may, for instance, perform quantitation of total and indicator bacteria to assess the effectiveness of pathogen-intervention processes such as hazard analysis and critical control points (HACCP)-based food safety procedures and other hygiene control measures. Typically, people seeking to determine the quantity of a pathogen rely on traditional methods for quantitation, such as most probable number (MPN) estimates based on serial culture dilution. Such approaches are often time consuming, tedious, and error-prone. In addition, such approaches may require specialized media and may take 24 hours or more to give results. Despite this, food, feed and water producers continue to rely on these methods for quantitation of total bacteria and indicator organisms (such as E. coli or coliforms).

### SUMMARY

The disclosure provides systems and methods for quantifying one or more target organisms, such as one or more species of a bacterial genus, present in a biological assay (e.g., a particular sample of food, feed, water, a raw material or corresponding environmental sample) using nucleic acid amplification assays and systems and methods for training a machine learning system to quantify target organisms present in a biological assay. The disclosure also provides methods for training a machine learning system to quantify target organisms present in inhibited biological assays.

An example system includes a detection device configured to amplify and detect a target nucleic acid associated with the target organism, such as a thermal cycler configured to carry out qPCR or other types of PCR. Some other such detection devices may be an isothermal device configured to carry out loop-mediated isothermal DNA amplification (LAMP). The detection device includes a reaction chamber configured to receive a sample having a quantity of the target nucleic acid and to amplify the target nucleic acid in the sample over a nucleic acid amplification cycle; and a detector, the detector configured to capture, during the nucleic acid amplification cycle, measurements representative of the quantity of the target nucleic acid present in the sample and to store the measurements in a data set, wherein the data set includes a first data subset, the first data subset including the measurements taken prior to a time Tₘₐₓ, wherein the time Tₘₐₓ corresponds to a time in the nucleic acid amplification cycle when the measurements reach a maximum amplitude, a second data subset, the second data subset including the measurements taken after the first point in time but before a second point in time in the nucleic acid amplification cycle, the second point in time occurring after Tₘₐₓ, and a third data subset, the third data subset including the measurements taken after the second point in time in the nucleic acid amplification cycle.

The system further includes a machine learning system configured to receive the first, second, and third data subsets and to apply a machine learning system to the data subsets. In some examples, the first, second and third data subsets include all the measurements in the data set. The machine learning system is trained to estimate a quantity of the target organism present in the sample based on the measurement samples in the first, second, and third data subsets.

An example method includes receiving a plurality of data sets, wherein each data set is associated with a biological assay, each data set including measurements, performed on the associated biological assay by a nucleic acid amplification device of a specified type and collected over at least a portion of a nucleic acid amplification cycle, of a target nucleic acid detected within the associated biological assay, wherein the target nucleic acid is associated with a target organism; labeling each data set with an estimate of the quantity of the target organism present within the associated biological assay; and training a machine learning system with the labeled data sets to estimate a quantity of the target organism within a biological assay based on tests performed on the target nucleic acid in the biological assay by nucleic acid amplification devices of the specified type.

An example non-transitory computer-readable medium includes instructions that, when executed by processing circuitry, cause the processing circuitry to receive a data set generated by amplifying a quantity of a nucleic acid in the sample over a nucleic acid amplification cycle, wherein the nucleic acid is associated with the target organism, the data set including measurements, collected during the nucleic acid amplification cycle, that are representative of the quantity of nucleic acid in the sample, wherein the data set includes a first data subset, the first data subset including the measurements taken prior to a time Tₘₐₓ, wherein the time Tₘₐₓ corresponds to a time in the nucleic acid amplification cycle when the measurements reach a maximum amplitude; a second data subset, the second data subset including the measurements taken after the first point in time but before a second point in time in the nucleic acid amplification cycle, the second point in time occurring after Tₘₐₓ; and a third data subset, the third data subset including the measurements taken after the second point in time in the nucleic acid amplification cycle; and to apply a machine learning system to the data subsets, wherein the machine learning system is trained to estimate a quantity of the target organism present in the sample based on the measurements present in the first, second, and third data subsets.

An example method of training a machine learning system to quantify a target organism present in a biological assay includes receiving data sets, each data set associated with a biological assay, each data set including data collected by a detector during nucleic acid amplification of a target nucleic acid within the associated biological assay across one or more nucleic acid amplification cycles, wherein the data collected by the detector includes activity measurements taken at different times during the one or more nucleic acid amplification cycles, wherein the target nucleic acid is associated with the target organism and wherein the biological assays include biological assays with different levels of inhibition; labeling each data set with an estimate of the quantity of the target organism present within the associated biological assay; and training a machine learning system to estimate a quantity of the target organism within a selected biological assay, the training based on the activity measurements stored in each data set and an estimate of the quantity of the target organism present in the biological assay associated with each respective data set.

An example system for quantifying a target organism present in a sample includes a detection device configured to amplify and detect a target nucleic acid associated with the target organism, the detection device comprising a reaction chamber configured to receive a biological assay having a quantity of the target nucleic acid and to amplify the target nucleic acid in the sample over a nucleic acid amplification cycle and a detector, the detector configured to capture, during the nucleic acid amplification cycle, activity measurements representative of the quantity of the target nucleic acid present in the sample taken at different times during the nucleic acid amplification cycle. The system further includes a machine learning system configured to receive the measurements and to apply the machine learning system to the measurements, wherein the machine learning system is trained using biological assays with different levels of inhibition to estimate a quantity of the target organism present in the sample based on the measurements, wherein training includes training the machine learning system to estimate a quantity of the target organism within a selected biological assay, the training based on the activity measurements stored in each data set and an estimate of the quantity of the target organism present in the biological assay associated with each respective data set.

Thus, in the systems and methods described herein, the data resulting from a biological assay may be collected and analyzed using machine learning systems, such as support vector machines, boosted decision trees, neural networks, and/or others. Such data may be used to train and build machine learning systems for particular pathogens. The machine learning systems, trained with one or more proper datasets, can examine much or all of a signal response in molecular diagnostic assays (e.g., qPCR and/or LAMP). Thus, such machine learning systems may be used both to extract non-linear relationships between variables and to estimate a quantity of organisms present in the original sample. Enabling quantitation of pathogens by applying trained machine learning systems to such molecular methods may yield results in a shorter period of time than traditional methods and/or may provide more accurate results at a lower cost relative to molecular methods that do not include the application of such trained machine learning systems.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1 is a block diagram illustrating an example system that includes a nucleic acid amplification device configured to amplify and detect a nucleic acid associated with a target organism and a user device configured to estimate a quantity of the target organism, in accordance with one aspect of the disclosure.

FIG. 2 is a block diagram illustrating an example system that includes an external device, such as a server, and an access point coupled to the nucleic acid amplification device of FIG. 1 via a network, in accordance with one aspect of this disclosure.

FIG. 3 is a schematic and conceptual diagram illustrating the example user device of FIG. 1, in accordance with one aspect of the disclosure.

FIG. 4 is a flow diagram illustrating example points for pathogen testing before, during, and/or after food or feed production, in accordance with one aspect of the disclosure.

FIG. 5 is a flow diagram illustrating an example technique for estimating a quantity of the target organism in a sample, in accordance with one aspect of the disclosure.

FIG. 6 illustrates real-time detection of nucleic acid amplification during a LAMP amplification cycle based on measurements of bioluminescence intensity over time, in accordance with one aspect of this disclosure.

FIG. 7 is a schematic drawing illustrating representative features of an example qPCR technique, in accordance with one aspect of this disclosure.

FIG. 8 illustrates limitations of the standard curve approach in quantifying pathogens when cell counts are used, in accordance with one aspect of this disclosure.

FIGS. 9A-9C are a flow diagrams illustrating example techniques for training a machine learning system and for using the trained machine learning system to estimate an initial quantity of a target organism in a sample, in accordance with one aspect of this disclosure.

FIG. 10 is a block diagram illustrating a device training system, in accordance with one aspect of this disclosure.

FIG. 11 illustrates a technique for training a machine learning model to estimate cell counts of target cells inoculated into a matrix and a technique for using the trained machine learning model to estimate cell counts in a matrix based on the trained model, in accordance with one aspect of this disclosure.

FIG. 12 illustrates log differences between cell count estimations made by a trained machine learning system and different cell counts of Salmonella cells inoculated into a poultry rinse matrix, in accordance with one aspect of this disclosure.

FIG. 13 illustrates log differences between cell count predictions made by a trained machine learning system and different cell counts of Salmonella cells inoculated into a poultry rinse matrix and also into a 1:10 dilution of the poultry rinse matrix, in accordance with one aspect of this disclosure.

FIG. 14 illustrates various metrics for measuring performance for regression used for cell count prediction using a variety of machine learning techniques, in accordance with aspects of this disclosure.

FIG. 15 is a conceptual drawing illustrating nucleic acid amplification in standard and inhibited samples during a LAMP amplification cycle, in accordance with one aspect of this disclosure.

FIG. 16 is a flow diagram illustrating an example technique for training a machine learning system to quantify target organisms in inhibited samples, in accordance with one aspect of this disclosure.

### DETAILED DESCRIPTION

In the following discussion, the term “food” also includes beverages. The term “water” includes drinking water, but the term “water” also includes water used in other situations that require quantitative measurements of one or more of the microorganisms in the water.

As noted above, food, feed and water producers use quantitative techniques to determine the quantity of microorganisms, such as bacterial pathogens, in food, feed (e.g., animal feed), water and corresponding processing environments. Quantitative techniques are used, for instance, to assess the effectiveness of pathogen-intervention processes used during food production. Such analysis may lead to more effective risk analyses and to the development of more effective ways to reduce the level of pathogens in the food, feed and water supply. The traditional methods discussed above for determining a quantity of pathogens in a biological assay are, however, time consuming, tedious, and error-prone. They may require specialized media and may take a day or more to give results.

Molecular methods (e.g., LAMP or PCR) may also be used to quantitate pathogens extracted from a sample. Molecular methods of pathogen quantification provide results in a shorter amount of time than more traditional methods (e.g., in hours rather than one or more days). In addition, they are not limited to quantification of total bacteria and indicator bacteria, but also may be used for quantifying specific bacteria, yeast, mold, or other pathogens. In practice, producers determine a pathogen quantity in a sample by extrapolating the quantity, based on test results from the sample, from a standard curve constructed from known nucleic acid concentrations. However, standard curves constructed from known nucleic acid concentrations may not correspond well to organism counts in samples collected from, for instance, production environments.

For instance, qPCR is widely used as a molecular method for detecting a variety of bacteria. qPCR may also be used for the absolute quantification of pathogens present in a given amount of sample. Standard curves containing known amounts of the target DNA (plasmids, genomic DNAs or other nucleic acid molecules) are run in parallel with the unknown samples. Based on the standard curve, the efficiency of the reaction and the dilution steps used for the nucleic acid extraction and analysis, the absolute number of pathogens in the unknown samples may be estimated. In these types of analysis, linear regression models are used, the efficiency of amplification becomes critical and standards need to be run with every run, adding to cost, time, possible contamination of samples. Furthermore, the standard curve approach has limited use when cell counts (not DNA) are being used. For these reasons, traditional methods are preferred over molecular methods for the quantification of microorganisms.

As noted above, assays based on molecular methods such as nucleic acid amplification ((e.g., LAMP or PCR) are highly efficient. They can, however, be affected by the presence of matrix-derived substances which can interfere or prevent the reaction from performing correctly, a process termed inhibition. In food production, matrix-derived substances, such as spices and environmental samples, may act as inhibitors that can interfere with nucleotide amplification assays such as PCR and LAMP, leading to false negative results.

It can be difficult to eliminate inhibition. Careful sample treatment may be used, for instance, to remove inhibitory substances. No sample treatment, however, can be relied on to completely remove inhibitory substances.

Amplification controls may also be used to control for inhibition. Such controls may be used, for instance, to verify that the assay has performed correctly. Typically, an internal amplification control (IAC) is a non-target DNA sequence present in the very same reaction as the sample or target nucleic acid extract. If it is successfully amplified to produce a signal, any non-production of a target signal in the reaction is considered to signify that the sample did not contain the target pathogen or organism. If, however, the reaction produces neither a signal from the target nor the IAC, it signifies that the reaction has failed, signally the absence of the target organism when, in fact, the target organism is present (i.e., a “false negative”). Detection of false negatives during the amplification cycle may be, therefore, critical for reliable testing.

The addition of amplification controls adds complexity and cost to molecular methods. It would be advantageous to eliminate the use of amplification controls when applying molecular methods to detect or quantify target organisms in a sample, even in the face of inhibition. Approaches for recognizing and correcting for inhibition are, therefore, presented below. These approaches may, for instance, be used to correct quantification in nucleotide amplification without the need for internal or external amplification controls.

The following disclosure describes systems and methods for quantitating pathogens in biological assays. The following disclosure further describes systems and methods for training and using machine learning systems in molecular methods of pathogen quantification, thereby improving the accuracy of pathogen quantification and reducing or eliminating the need for preparing and using standard curves with every run. In some example methods described herein, LAMP bioluminescent assays and/or PCR assays (e.g., qPCR assays) may be used in a training run to amplify a target nucleic acid (e.g., a nucleic acid associated with a target organism) present in a sample in a known initial quantity and to detect light generated within the sample during amplification of the target nucleic acid. In other example methods described herein, assays such as nicking-enzyme amplification reaction (NEAR), helicase-dependent amplification (HDA), nucleic acid sequence-based amplification (NASBA), or transcription-mediated amplification (TMA) assays may be used.

Any suitable variation on such assays may be used. Variations on a traditional LAMP assay that may be used may include colorimetric LAMP (cLAMP) assays, in which pH changes driven by the accumulation of protons during LAMP can be visualized via observation of color changes of a pH-sensitive colorimetric dye that occur with nucleic acid amplification. Other such variations may include turbidity-LAMP assays, in which formation of magnesium pyrophosphate during LAMP results in turbidity that increases in correlation with nucleic acid yield and that can be quantified in real-time. Materials and methods used in such variations on traditional LAMP assays, and/or on PCR assays, may be understood by those of skill in the art and thus are not described in detail here. It should be understood that example nucleic acid amplification techniques and variations thereon described herein are not intended to be limiting. Instead, any suitable nucleic acid amplification technique may be used in the techniques described herein, such as in a training run to amplify a target nucleic acid.

Data from the training run may be fed into a machine learning system to train the machine learning system. The trained machine learning system then may be used to estimate an unknown initial quantity of the target organism present in a sample, such as a food sample, feed sample, water or environmental sample from a food or feed processing environment. In other example methods described within, LAMP bioluminescent assays and/or PCR assays (e.g., qPCR assays) may be used in a training run to amplify a target nucleic acid (e.g., a nucleic acid associated with a target organism) present in a series of samples having known initial quantities of the target organism. The method collects data for each sample representative of light generated within the sample during amplification of the target nucleic acid and associates the collected data with known quantities of the target nucleic acid, or with known quantities of the organism being detected. Data from the training run is then fed into a machine learning system to train the machine learning system. The trained machine learning system may then be used to estimate an unknown initial quantity of the target organism present in a sample, such as a food sample, feed sample, water or environmental sample from a food or feed processing environment.

In yet other example methods described within, LAMP bioluminescent assays and/or PCR assays (e.g., qPCR assays) may be used to obtain data corresponding to samples collected from a particular environment (e.g., a poultry processing plant or a cheese factory). The samples are reviewed using traditional quantitation methods and each sample is labeled with a quantity value determined via one or more of the traditional methods. The data from the labeled samples is then fed into a machine learning system to train the machine learning system for that particular environment. The trained machine learning system may then be used to better estimate an unknown initial quantity of the target organism and/or nucleic acid present in a sample, such as a food sample, feed sample, water or environmental sample from the particular environment.

It should be noted that while in some examples nucleic acids associated with a target organism may be described herein as being DNA, in other examples, a nucleic acid associated with a target organism may be an RNA. In such other examples, an amplification technique such as quantitative reverse transcription PCR (RT-qPCR) and reverse transcription LAMP (RT-LAMP) on total RNA or mRNA of a sample may be used in a method of training a machine learning system to estimate an initial quantity of a target organism in a sample and/or in applying such a trained machine learning system.

Each machine learning system is based on at least one model. The model may be a regression model based on techniques such as, for example, support vector regression, random forest regression, linear regression, ridge regression, logistic regression, Lasso, or nearest neighbor regression. Or the model may be a classification model based on techniques such as, for example, support vector machines, decision tree and random forest, linear discriminant analysis, neural networks, nearest neighbor classifier, stochastic gradient descent classifier, gaussian process classification, or naïve Bayes. Both types of models rely on the use of labeled data sets to train the model.

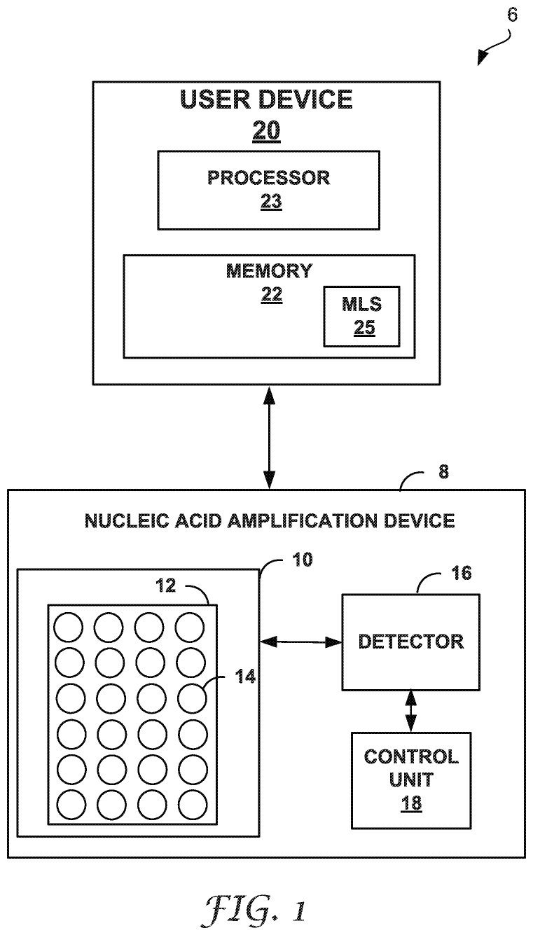

FIG. 1 is a block diagram illustrating an example system that includes a nucleic acid amplification device configured to amplify and detect a nucleic acid associated with a target organism and a user device configured to estimate a quantity of the target organism, in accordance with one aspect of the disclosure. Nucleic acid amplification device 8 is configured to amplify and detect a target nucleic acid, in accordance with one aspect of the disclosure. Nucleic acid amplification device 8 includes a reaction chamber 10 configured to amplify the target nucleic acid. In one example approach, as shown in FIG. 1, reaction chamber 10 includes a block 12 that may be heated and/or cooled via a heat source such as a Peltier system. As illustrated in FIG. 1, block 12 defines a plurality of wells 14, each of which may be dimensioned to receive a reaction vessel, which may be any suitable plastic tube configured for use in nucleic acid amplification assays. Nucleic acid amplification device 8 further includes a detector 16 and a control unit 18. Detector 16 may be configured to capture light within reaction chamber 10 under control of control unit 18. For example, detector 16 may be configured to capture a data set including time-series measurement samples of light emitted by a light-emitting species within sample contained within a reaction vessel received within one of wells 14 during one or more nucleic acid amplification cycles. In some examples, the sample may include a target nucleic acid and the light-emitting species, the latter of which may emit light in a stoichiometric relationship with the target nucleic acid such that the light emitted by the light-emitting species increases with an increase in the quantity of replicated target nucleic acid in the sample.

In some examples, nucleic acid amplification device 8 may be any suitable nucleic acid amplification device configured for LAMP (e.g., traditional LAMP assays, or cLAMP, turbidity LAMP, or other variations on traditional LAMP assays). In examples in which light is emitted by a light-emitting species captured by detector 16, the light may be bioluminescence, fluorescence or light of any visible color. In examples in which a turbidity LAMP technique is used, the detector may measure at least one of absorbance, transmittance, or reflectance. Additionally, or alternatively, nucleic acid amplification device 8 may be any suitable nucleic acid amplification device configured for qPCR or any other nucleic acid amplification technique (e.g., NEAR, HDA, NASBA, TMA, or others). In some such other examples, light emitted by the light-emitting species and captured by detector 16 may be fluorescence.

In some of the example methods described herein for training a machine learning system to quantify a target nucleic acid present in a biological assay (e.g., carried out in a reaction vessel using nucleic acid amplification device 8), nucleic acid amplification device 8 may be a nucleic acid amplification device of a specified type. For example, nucleic acid amplification device 8 may include one or more specific features and/or may be a specific model of a nucleic acid amplification device from a specified manufacturer. In some such examples, a trained machine learning system resulting from such methods may be tailored to the specified type of nucleic acid amplification device, which may enhance the accuracy of the trained machine learning system. Nucleic acid amplification devices having any suitable configuration may be used. For example, a nucleic acid amplification device may include a rack (e.g., a spinning rack) configured to receive reaction vessels instead of a block. In some such examples, the reaction vessels may be capillaries or more traditionally-configured tubes. In some examples, a detector 16 of a nucleic acid amplification device may be position above the reaction vessels or in any suitable position. Thus, the configuration of nucleic acid amplification device described herein is not intended to be limiting but to illustrate an example.

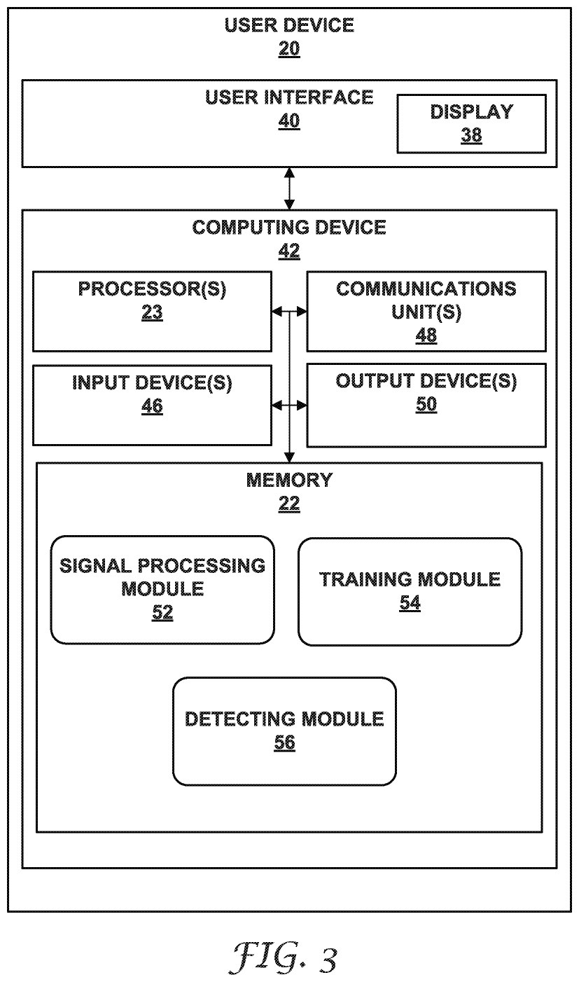

The example system of FIG. 1 further includes user device 20, which may include a processor 23 and a memory 22 used to store parameters representing one or more trained machine learning systems 25. In one example approach, user device 20 receives a data set from control unit 18 for each sample tested. In some such example approaches, each data set includes data representing a quantity of light received by detector 16 at specific times during the amplification cycle of the given sample. As further discussed below with respect to FIG. 3, user device 20 may be a device such as a computer workstation, tablet, or other such user device co-located with nucleic acid amplification device 8 in a user's laboratory. Nucleic acid amplification device 8 may be configured to transmit the data set from control unit 18 to user device 20, such as via any suitable wired connection (e.g., metal traces, fiber optics, Ethernet, or the like), a wireless connection (e.g., personal area network, local area network, metropolitan area network, wide area network, a cloud-based system, or the like), or a combination of both. For example, user device 20 may include a communications unit that includes a network interface card, such as an Ethernet card, an optical transceiver, a radio frequency transceiver, a Bluetooth® interface card, WiFi™ radios, USB, or any other type of device that can send and receive information to and from nucleic acid amplification device 8.

In some example approaches, processor 23 may be configured to apply a trained machine learning system 25 stored in memory 22 to the data set and to estimate a quantity of a target organism present in the biological assay as a function of the data set. In some examples, processor 23 may store the estimated quantity of the target organism, such as in association with other data pertaining to the biological assay. The estimated quantity of the target organism may be compared to a corresponding threshold value in a limit test to determine whether the sample passes or fails the limit test. The threshold value may, in some such example approaches, be a value associated with one or more regulatory standards, industry practices, or associated intervention processes. For example, the estimated quantity of the target organism in a sample may help enable evaluation of effectiveness of intervention procedures designed to improve process efficiency and/or reduce pathogen levels in food products, feed products, water and/or corresponding preparation environments.

In this manner, systems and methods that include applying a trained machine learning system to a data set associated with an amplified sample of a target nucleic acid to estimate a quantity of the target organism in the sample may help address public health issues associated with pathogens. For example, since the systems and methods for nucleic acid quantitation described herein provide quantity values more quickly than traditional approaches to pathogen quantitation, such systems and methods may make pathogen quantitation more accessible to the food industry. This increased accessibility may be used by the food industry, for instance, to obtain a more nuanced understanding of pathogen presence than can be obtained simply by detecting the presence or absence of the pathogen. The increased accessibility may also be used to support limit testing in pathogen analysis, as one goal of limit testing is to detect foodborne pathogen concentrations that meet or exceed a threshold concentration and limit the release of products that may negatively impact public health.

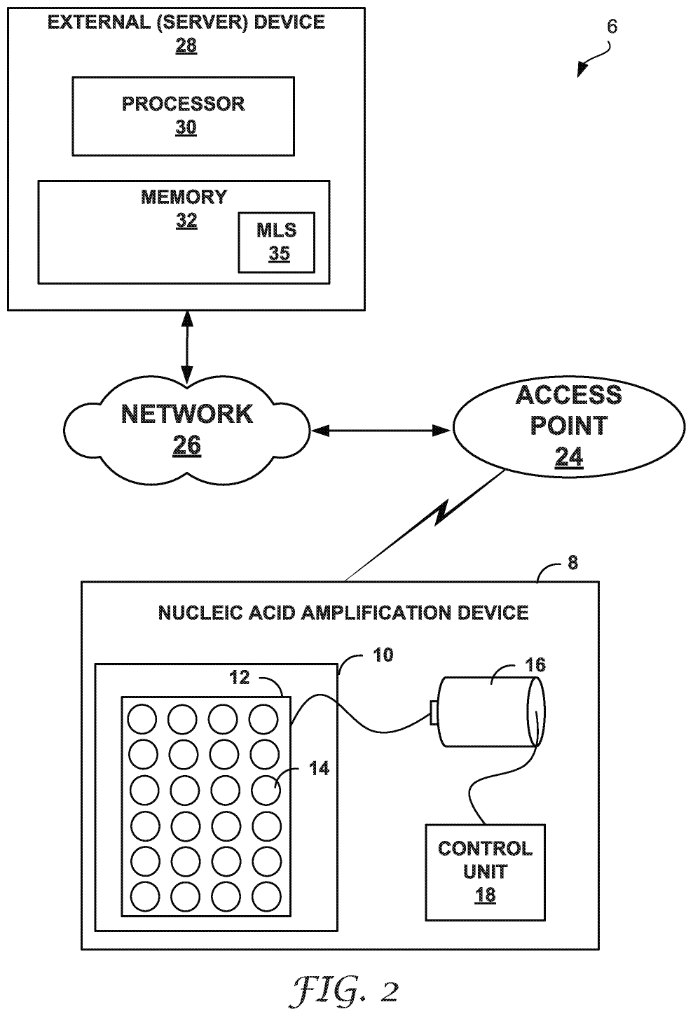

FIG. 2 is a block diagram illustrating an example system 6 that includes the nucleic acid amplification device 8 of FIG. 1, an external device, such as a server, a network and an access point coupling the nucleic acid amplification device to the external device via the network, in accordance with one aspect of this disclosure. In one example, as shown in FIG. 2, system 6 may include an access point 24, a network 26, and one or more external devices, such as an external device 28 (e.g., a server), which may include a processing circuitry 30 and/or memory 32. In the example shown in FIG. 2, nucleic acid amplification device 8 may use communication circuitry (not shown) used to communicate with access point 24 via a wireless connection. Access point 24 then conveys the information received from nucleic acid amplification device 8 to external device 28 through network 26 via a wired connection and conveys the information received from external device 28 through network 26 to nucleic acid amplification device 8 via the wireless connection.

Access point 24 may comprise a processor that connects to network 26 via any of a variety of connections, such as telephone dial-up, digital subscriber line (DSL), or cable modem, or other suitable connections. In other examples, access point 24 may be coupled to network 26 through different forms of connections, including wired or wireless connections. In some examples, access point 24 may be a user device, such as a computer workstation or tablet that may be co-located with nucleic acid amplification device 8 and the user. Nucleic acid amplification device 8 may be configured to transmit data to access point 24, such as data sets described above with respect to FIG. 1. In addition, access point 24 may interrogate nucleic acid amplification device 8, such as periodically or in response to a command from a user or from network 26, in order to retrieve data sets pertaining to one or more biological assays, or to retrieve other information stored in a memory (not shown) of nucleic acid amplification device 8. Access point 24 may then communicate the retrieved data to external device 28 via network 26.

In some examples, memory 32 of external device 28 may be configured to provide a secure storage site for data collected from access point 24 and/or nucleic acid amplification device 8. In some examples, memory 32 stores parameters representing one or more trained machine learning systems 35. In some examples, external device 28 may assemble the data in web pages or other documents for viewing by users via access point 24 or one or more other computing devices of the system of FIG. 2. In this manner, the system of FIG. 2 may enable remote (e.g., cloud-based) storage and access of data associated with a user's testing of food or feed products and/or of corresponding production environments. Such systems may be customized to meet a particular user's data storage and/or access needs.

FIG. 3 is a schematic and conceptual diagram illustrating features of user device 20 of FIG. 1, in accordance with one aspect of the disclosure. Although FIG. 3 is described with respect to user device 20 of FIG. 1, one or more components of user device 20 described herein may be functionally and/or structurally similar to one or more components of access point 24 and/or external device 28 illustrated in FIG. 2. In one example approach, user device 20 includes user interface 40 and computing device 42. User interface 40 may include display 38, a graphical user interface (GUI), a keyboard, a touchscreen, a speaker, a microphone, or the like.

Computing device 42 includes one or more processors 23, one or more input devices 46, one or more communications units 48, one or more output devices 50, and memory 22. In some examples, computing device 42 and user interface 40 are components of the same device, such as a computer workstation, a tablet, or the like. In some such examples, user interface 40 may include one or more of input devices 46. In other examples, computing device 42 and user interface 40 are separate devices such that user interface 40 does not necessarily include one or more of input devices 46.

One or more processors 23 of computing device 42 are configured to implement functionality, process instructions, or both for execution within computing device 42. For example, processors 23 may be capable of processing instructions stored within memory 22, such as instructions for applying a trained machine learning system to a data set to estimate an initial quantity of a target nucleic acid or a target organism present in a sample. Examples of one or more processors 23 may include, any one or more of a microprocessor, a controller, a digital signal processor (DSP), an application specific integrated circuit (ASIC), a field-programmable gate array (FPGA), or equivalent discrete or integrated logic circuitry.

In some examples, computing device 42 may utilize one or more communications units 48 to communicate with one or more external devices (e.g., external device 28 of FIG. 2 and/or nucleic acid amplification device 8) via one or more networks, such as one or more wired or wireless networks. Communications units 48 may include a network interface card, such as an Ethernet card, an optical transceiver, a radio frequency transceiver, or any other type of device configured to send and receive information. Communications units 48 may also include WiFi™ radios or a Universal Serial Bus (USB) interface.

In some examples, one or more output devices 50 of computing device 42 may be configured to provide output to a user using, for example, audio, video or tactile media. For example, output devices 50 may include display 38 of user interface 40, a sound card, a video graphics adapter card, or any other type of device for converting a signal into an appropriate form understandable to humans or machines, such as a signal associated with information pertaining to a status, outcome, or other aspect of one or more data sets resulting from amplification cycles carried out by nucleic acid amplification device 8 analyzed by a trained machine learning system. In some example approaches, user interface 40 includes one or more of output devices 50 employed by computing device 42.

Memory 22 of computing device 42 may be configured to store information within computing device 42 during operation. In some examples, memory 22 may include a computer-readable storage medium or computer-readable storage device. Memory 22 may include a temporary memory, meaning that a primary purpose of one or more components of memory 22 may not necessarily be long-term storage. Memory 22 may include a volatile memory, meaning memory 22 does not maintain stored contents when power is not provided thereto. Examples of volatile memories include random access memories (RAM), dynamic random-access memories (DRAM), static random-access memories (SRAM), and other forms of volatile memories known in the art. In some examples, memory 22 may be used to store program instructions for execution by processors 23, such as instructions for applying a trained machine learning system to a data set received from nucleic acid amplification device 8 via one or more communications units 48. Memory 22 may, in some examples, be used by software or applications running on computing device 42 to temporarily store information during program execution.

In some examples, memory 22 may further include a signal processing module 52, a training module 54, and a detecting module 56. In some such examples, detecting module 56 includes a machine learning system (such as machine learning systems 25 and 35) that, when trained, estimates the concentration of target organisms in a sample. In one such example approach, training module 54 receives data sets of assays with known cell concentrations collected by a nucleic acid amplification device 8 over one or more amplification cycles and uses the data sets to train detecting module 56 to estimate the concentration of target organisms in a sample.

In some examples, memory 22 may include non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In one such example approach, signal processing module 52 may be configured to analyze data received from nucleic acid amplification device 8, such as a data set capture by detector 16 and comprising time-series measurement samples of the light emitted by light-emitting species within a sample during an amplification cycle, and process the data to improve the quality of the sensor data.

Computing device 42 may also include additional components that, for clarity, are not shown in FIG. 3. For example, computing device 42 may include a power supply to provide power to the components of computing device 42. Similarly, the components of computing device 42 shown in FIG. 3 may not be necessary in every example of computing device 42.

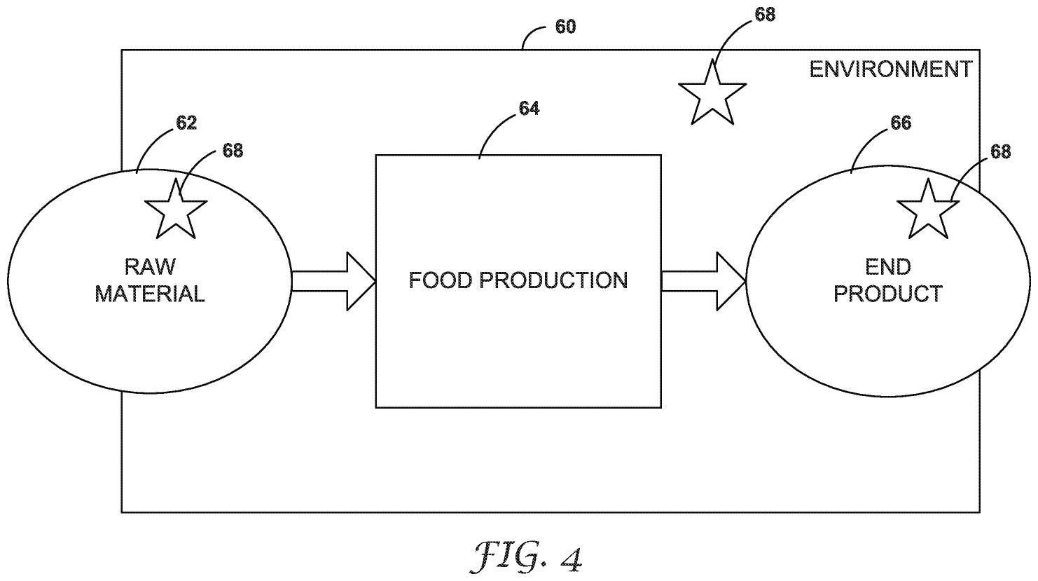

FIG. 4 is a flow diagram illustrating example points for pathogen testing before, during, and/or after food or feed production, in accordance with one aspect of the disclosure. As illustrated in FIG. 4, food production environment 60 may include raw material 62. Food production processes 64 that process raw material 62 and produce end product 66 may take place within food production environment 60. In some examples, production processes 64 may take place entirely within food production environment 60, whereas raw material 62 may enter food production environment 60 from outside of food production environment 60 at the beginning of the processes illustrated in FIG. 4. In some examples, food production environment 60 may be an environment in which food or feed materials are harvested, such as a greenhouse or field in which such materials are grown. In some examples, samples from food production environment 60 may be water samples from water sources within the food production environment 60, such as sources of water used for washing and/or cooking.

Raw material 62 may acquire pathogens from outside food production environment 60 and introduce such pathogens into food production environment 60 as or after raw material 62 is introduced into food production environment 60. Thus, to help reduce foodborne illness caused by pathogens, there is an increased trend in pathogen testing of raw materials (e.g., raw material 62) and food production environments (e.g., food production environment 60). Moreover, pathogen testing of raw material 62 may help prevent pathogen contamination of end product 66 (or of other end products) by identifying contamination before raw material enters food production environment 60 such that entrance of contaminated raw materials into food production environment 60 may be avoided.

End product 66 may be located within environment 60 for a period of time prior to shipment out of environment 60, such as before, during, and after packaging. End product 66 may acquire pathogens from food production environment 60, such as pathogens introduced by raw material 62 or from other sources within food production environment 60. However, as discussed above, traditional methods of pathogen quantification may be significantly time consuming, taking one or more days to yield results, and molecular methods of pathogen quantification have not yet gained widespread use. In some instances, the time required for traditional methods of pathogen quantification may limit food processing rates. Moreover, due to the time requirement, such traditional methods provide pathogen assessment only as current as the time the sample was taken, which may not provide an accurate assessment of a current state of a material, environment, or product. Thus, at least due to the time advantage of the molecular methods for pathogen quantification described herein, pathogen testing of raw material 62, food production environment 60, and/or end product 66 (e.g., as part of a release test), such as at test points 68, according to such methods that may provide more up-to-date assessments, which ultimately may help prevent the release of contaminated end products to the public.

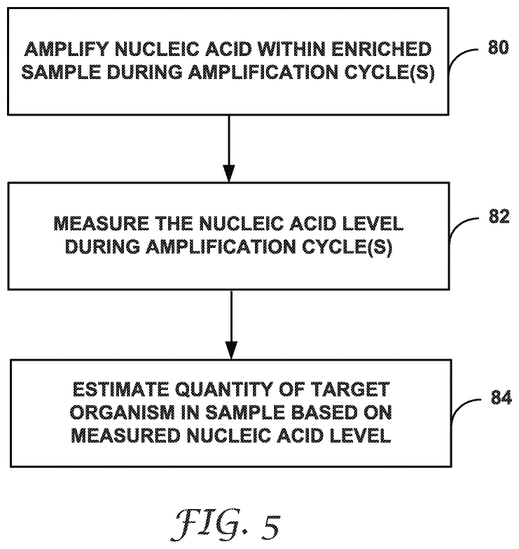

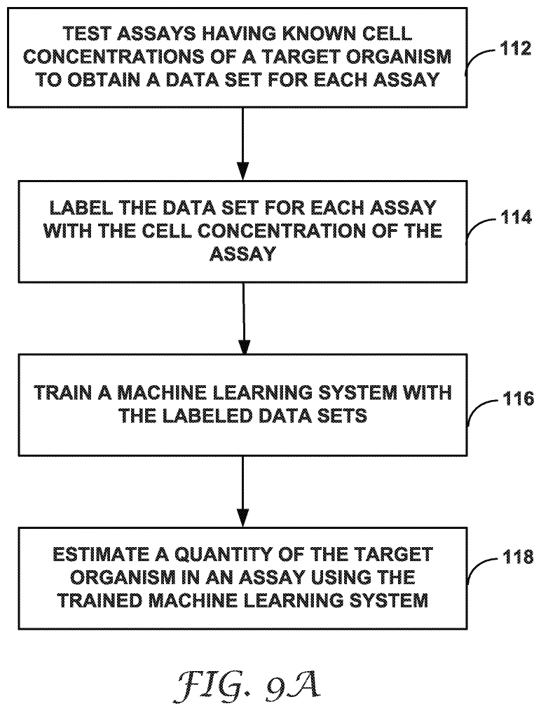

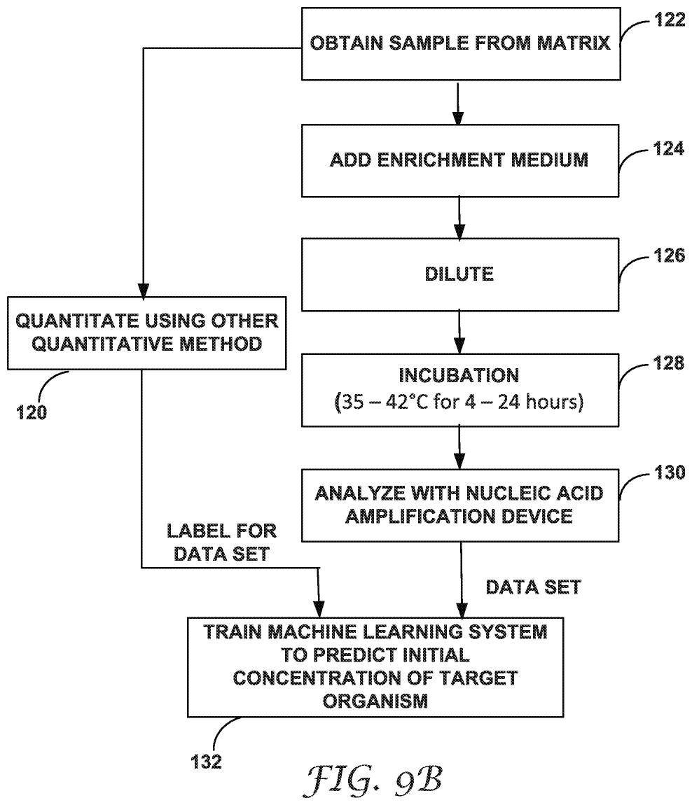

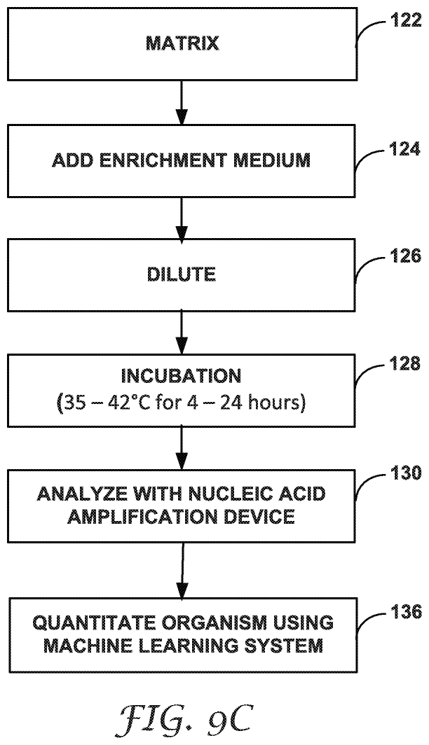

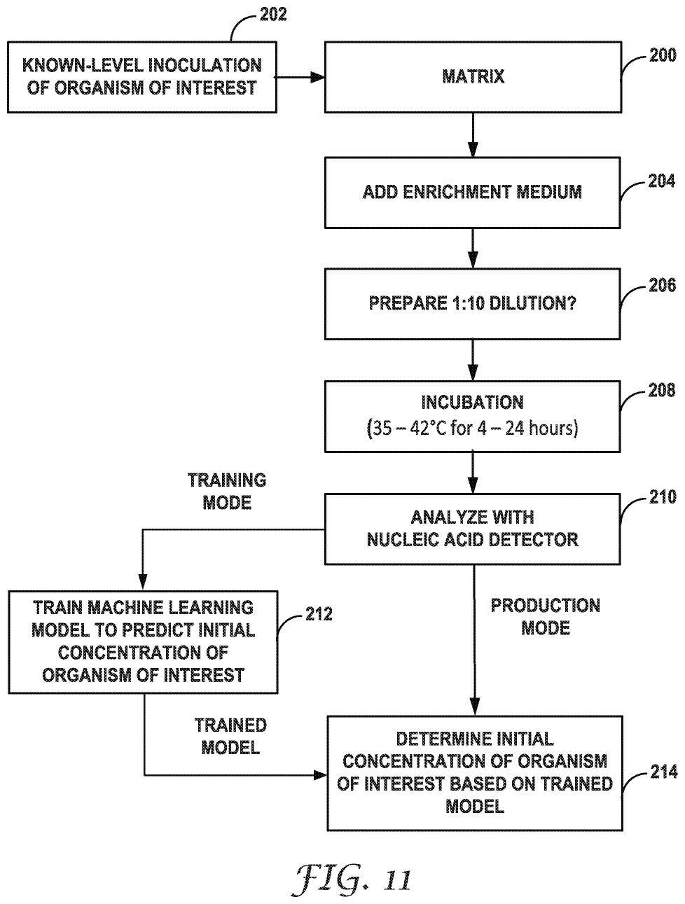

FIG. 5 is a flow diagram illustrating an example technique for estimating a quantity of the target organism in a sample, in accordance with one aspect of the disclosure. The example approach of FIG. 5 may be carried out using a nucleic acid amplification device such as nucleic acid amplification device 8 of the systems of FIGS. 1 and 2. As described above with respect to FIG. 1, nucleic acid amplification device 8 may be a nucleic acid amplification device of any suitable type and may be configured to carry out any suitable nucleic acid amplification technique, such as LAMP or PCR. Although described in the context of the systems of FIG. 1, the example technique of FIG. 5 may be carried out using any suitable nucleic acid amplification device and computing device. More specific aspects and examples of the technique generally illustrated in FIG. 5 are described below with respect to FIGS. 9A-9C and 11.

In the example approach of FIG. 5, nucleic acid amplification device 8 amplifies a target nucleic acid within an enriched sample within reaction chamber 10 (80). In some examples, the sample may be derived from food production environment 60, raw material 62, or end product 66 as described above with respect to FIG. 4. Nucleic acid extracted from the sample may be placed within a reaction vessel (e.g., a PCR tube) and a light-emitting species that emits light in a stoichiometric relationship with the target nucleic acid, which may be a DNA sequence associated with a target organism (e.g., a bacterial genus or species). In some examples, the sample may be an enriched sample derived from a sample of food or feed raw material, end product, water or production environment. For example, the sample placed in the reaction vessel may be an enriched sample from a culture derived from the initial sample. In some such examples, the estimated quantity of the organism may be an estimated initial quantity of the organism. In some examples, such reaction vessel containing a sample and a light-emitting species collectively may be referred to herein as a “biological assay.” Detector 16 of nucleic acid amplification device captures a data set comprising time-series measurement samples of the light emitted by the light-emitting species over one or more amplification cycles and transmits the data set to computing device 42 of user device 20, a computing device of access point 24, or any other suitable computing device (82).

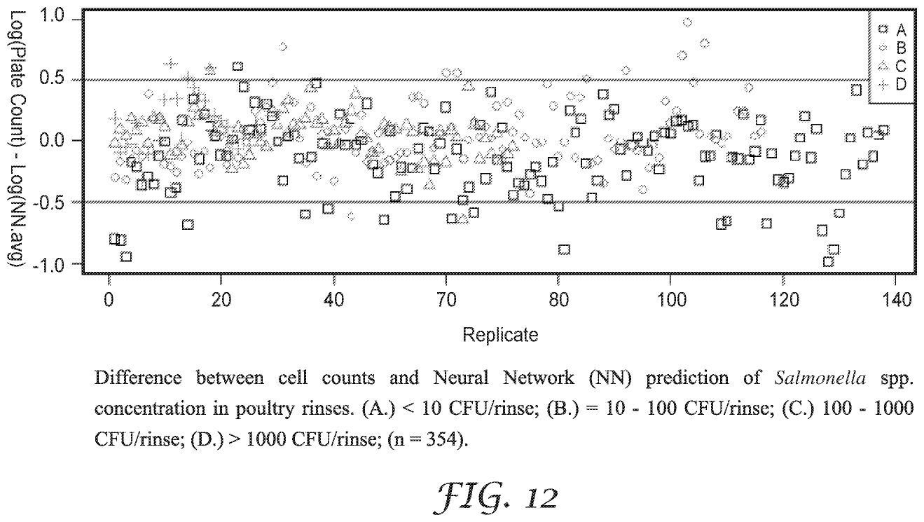

In the example of user device 20, one or more of processors 23, signal processing module 52, and/or other components of computing device 42 may apply a trained machine learning system to the data set to estimate the quantity of the target organism in the sample (84). In some examples, the data set may include one or more data subsets associated with one or more different portions or phases of the amplification cycle, such as one or more portions or phases before, during, and/or after a peak amplitude of light emitted over the amplification cycle. Including data subsets from such different portions or phases of the amplification cycle may contribute to the accuracy with which the trained machine learning system may estimate the quantity of the target organism in the sample, as further described below with respect to FIGS. 11 and 12.

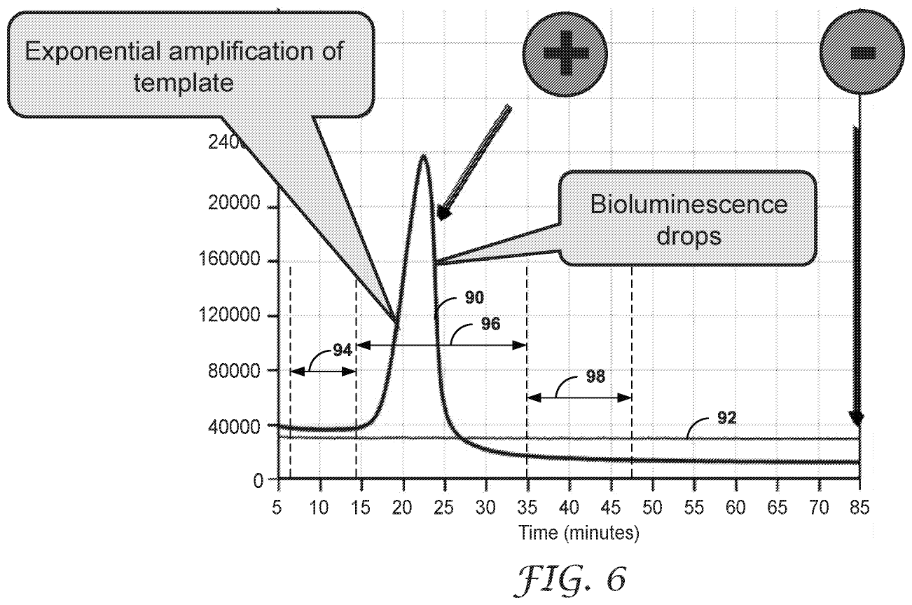

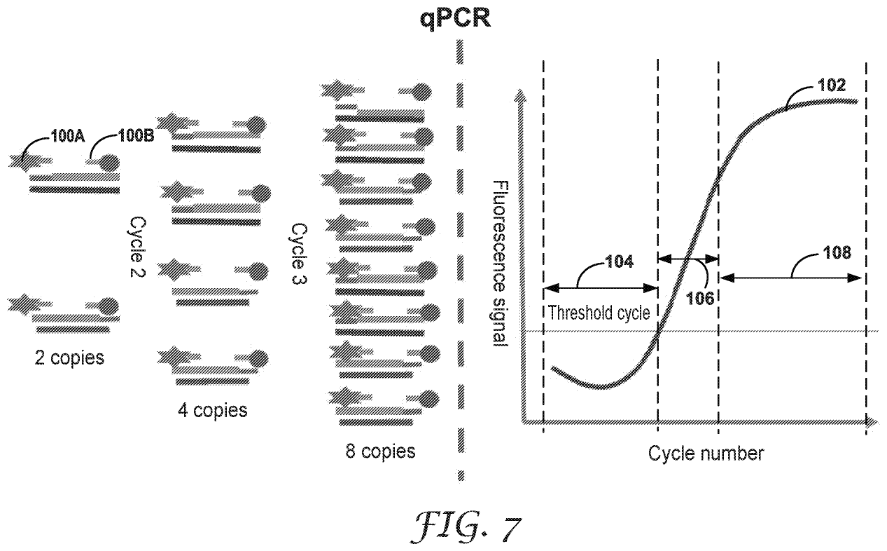

FIGS. 6 and 7 are conceptual drawings illustrating representative features of example nucleic acid amplification techniques that may be used with the systems and methods described herein. Technical aspects of an example LAMP technique are described below with respect to FIG. 6, such as to the extent that such technical aspects may be relevant to arriving at the example of FIG. 6. FIG. 7 illustrates aspects of an example qPCR technique that may be used with the systems and methods described herein. Technical aspects of an example qPCR technique are discussed below with respect to FIG. 7, such as to an extent that such technical aspects may be relevant to arriving at the example of FIG. 7. However, it should be understood that the systems and methods described herein may be used with any suitable nucleic acid amplification technique and device, and are not limited to the particular examples described with respect to FIGS. 6 and 7.

LAMP uses strand-displacing Bst DNA polymerase and four to six primers to produce continuous DNA amplification at a constant temperature (i.e., under isothermal conditions). In LAMP techniques, amplification and detection of a target nucleic acid can be completed in a single step, by incubating a mixture of a sample, primers, a DNA polymerase with strand displacement activity, and substrates at a constant temperature (about 60 to 65° C.). In some examples, LAMP may provide high amplification efficiency, with DNA being amplified 10⁹-10¹⁰ times in 15-60 minutes. Because of its high specificity, the presence of amplified product can indicate the presence of target gene.

In LAMP, four different primers recognize six distinct regions in a template (i.e., target) DNA sequence and two loop primers recognize two additional sites in corresponding single stranded loop regions during LAMP. The four different primers that recognize the six distinct regions of the target DNA may include a Forward Internal Primer (FIP), a Forward Outer Primer (F3; aka FOP), a Backward Inner Primer (BIP), and a Backward Outer Primer (B3; aka BOP). The two loop primers include Forward Loop Primer (FLP) and Backward Loop Primer (BLP). In contrast, PCR and qPCR each use non-strand displacing Taq DNA polymerase and two corresponding primers, a forward primer and a backward primer to recognize two distinct regions. In addition, qPCR uses a probe (e.g., a fluorescence-emitting molecular beacon probe, a fluorescence-emitting hydrolysis probe, a primer carrying a fluorescence-emitting probe element, or another suitable probe that includes a fluorescent moiety) having specificity to a third distinct region.

The two loop primers FL and BL may bind to additional sites during LAMP and accelerate reactions. For example, primers containing sequences complementary to the single stranded loop region (either between the B1 and B2 regions, or between the F1 and F2 regions) on the 5′ end of a dumbbell-like structure formed during LAMP may provide an increased number of starting points for DNA synthesis during a LAMP technique. For example, an amplified product containing six loops (not shown) may be formed during LAMP. In example techniques in which loop primers FL and BL are not used, four out of six of such loops would not be used. Through the use of loop primers, all the single stranded loops can be used as starting points for DNA synthesis, thereby reducing amplification time. For example, the time required for amplification with loop primers may be about one-third to about one-half of the time required for amplification in examples in which loop primers are not used. In some examples, with the use of loop primers, amplification may be achieved within 30 minutes.

FIG. 6 illustrates real-time detection of nucleic acid amplification during a LAMP amplification cycle based on measurements of bioluminescence intensity over time, in accordance with one aspect of this disclosure. In an example LAMP technique, isothermal DNA amplification releases pyrophosphate (PPi) as a byproduct. The byproduct PPi is then converted to adenosine triphosphate (ATP) by the enzyme ATP-sulfurylase in the presence of adenosine 5′-phosphosulfate. In one such example approach, a biological assay having a sample being analyzed for a target nucleic acid may be adapted to include the luciferase enzyme and its substrate luciferin, the latter of which may be used as the light-emitting species in the example systems and methods described herein. Since ATP is a co-factor for the reaction of the luciferase enzyme and bioluminescence-producing luciferin, the conversion of PPi to ATP during an amplification cycle of a LAMP technique drives the emission of bioluminescence. This emission of bioluminescence may be detected by a detector of a nucleic acid amplification device configured for LAMP, such as detector 16 of nucleic acid amplification device 8 of FIGS. 1 and 2, and data representing time-series measurements of the bioluminescence are stored as a data set. In some examples, the mechanism for generating light during a LAMP technique illustrated in FIG. 6 may provide one or more other benefits, such as enabling real-time detection of nucleic acid amplification occurring during the LAMP amplification cycle over a relatively short period of time, such as about 15 minutes.

Time-series measurements of relative light units (RLU) emitted by the light-emitting species (e.g., luciferin) in a biological assay containing the target nucleic acid are depicted in curve 90. Time-series measurements of relative light units (RLU) emitted by the light-emitting species (e.g., luciferin) in a control not containing the target nucleic acid are depicted in baseline curve 92. As shown by curve 90, exponential amplification of the target nucleic acid during the LAMP amplification cycle produces a bioluminescence signal having both a rapid increase in RLU and a rapid decrease in RLU. In such examples, the time-to-peak RLU emission corresponds to the quantity of the target organism. For example, a relatively greater quantity of the target organism may produce a shorter time-to-peak RLU emission. Thus, one or more aspects of curve 90, such as the time-to-peak or amplitude, may be used in training a machine learning system to estimate a quantity of a target organism in a sample.

In some examples, the data set used to train a machine learning system such as a neural network includes data captured as a set of time-series measurement samples of bioluminescence captured across the entirety of the amplification cycle. In one such example, luminescence measurements are taken approximately every 5 seconds, which may be accumulated as measurements at 10, 15, 20, and/or 25 second intervals across the amplification cycle for reporting purposes.

In some example approaches, the data set used to train a machine learning system such as a neural network includes time-series measurement samples of bioluminescence taken across the entirety of the nucleic acid amplification cycle. In other example approaches, the training data set includes measurements taken during one or more of a first phase 94 of the amplification cycle, a second phase 96 of the amplification cycle and a third phase 98 of the amplification cycle. In some such examples, a machine learning system may be trained to estimate a quantity of the target organism present in a sample based on samples in each of the first, second, and third data subsets, based on the data set of samples taken across the entire amplification cycle, or based just on samples in the second subset. In one such example approach, the samples from the second subset include a sample taken at Tₘₐₓ, where Tₘₐₓ is the time during the nucleic acid amplification cycle that the maximum amplitude of the target nucleic acid is detected. Again, samples may be taken approximately every 5 seconds, which may be accumulated to measurements from about 10, 15, 20, and/or 25 seconds across the amplification cycle for reporting purposes. Training the machine learning system based in part on data subsets not associated with peak amplification may provide more robust training than training based only on one or more data subsets associated with peak amplification, which in turn may enhance the ability of the trained machine learning system to accurately estimate an unknown quantity of the target organism.

A detector, such as detector 16 of nucleic acid amplification device 8, may capture a data set that includes time-series measurement samples of the light emitted by the light-emitting species during the amplification cycle as depicted in curve 90 and transmit the data set to a computing device (e.g., computing device 42), which may apply a trained machine learning system. In this manner, the mechanism for generating light during a LAMP technique described with respect to FIG. 6 may enable a user to obtain an estimated quantity of the target organism in the sample much sooner than may be practicable using traditional pathogen quantitation methods.

In PCR, DNA extension is limited to a specific period of each thermocycle (i.e., amplification cycle). In PCR, the presence of inhibitors can prevent the polymerase from extending the DNA in the time allowed, which may result in incomplete amplification products and may prevent the detection of the target organism. PCR's temperature cycling and the association and disassociation of the polymerase from the DNA template during the denaturation step provides many opportunities for inhibitors to interfere. Inhibition may be less likely to occur in LAMP techniques than in PCR- and Immunoassay-based systems. Also, PCR may be more likely to be subject to interference by the natural fluorescence of some food samples and enrichment media. Thus, use of LAMP techniques may provide one or more benefits over the use of PCR techniques in the systems and methods described herein. However, as discussed above, the use of PCR techniques in conjunction with the systems and methods described herein may provide one or more benefits over traditional pathogen quantitation methods in other examples.

FIG. 7 illustrates detection of nucleic acid amplification during an example qPCR technique across multiple PCR cycles based on measurements of fluorescence intensity over time, in accordance with one aspect of this disclosure. In some such examples, a light-emitting species may be a fluorescence-emitting hydrolysis probe, such as a TaqMan hydrolysis probe (available from Thermo Fisher Scientific). During PCR, 5′-3′ exonuclease activity of the Taq polymerase cleaves the probe into two portions, 100A and 100B, during hybridization to a complementary target DNA sequence. Cleavage of the hydrolysis probe produces a fluorescence signal, represented in FIG. 7 by curve 102.

As shown by curve 102, amplification of the target nucleic acid during the PCR run including multiple amplification cycles produces a fluorescence signal. Curve 102 may include several portions or phases that reflect corresponding portions or phases of amplification of the target nucleic acid. For example, curve 102 may include a first portion 104 corresponding to an initiation phase of amplification, during which the fluorescence signal may remain below a threshold. Curve 102 further may include a second portion 106 corresponding to an exponential phase of amplification, during which the fluorescence exceeds the threshold and increases exponentially. Finally, curve 102 may include a third portion 108 corresponding to a plateau phase of amplification, during which the fluorescence remains above threshold and slowly increases over additional amplification cycles.

As with the example LAMP technique of FIG. 6, a machine learning system may be trained to estimate a quantity of the target organism present in a sample based on each of the first, second, and third data subsets corresponding to respective ones of the first, second, and third phases of the fluorescence signal as noted above. The machine learning system may also be trained to estimate a quantity of the target organism present in the sample based on a data set of fluorescence signal measurements collected across the entirety of the amplification cycle. Training the machine learning system based in part on data subsets not associated with the exponential amplification phase of a PCR run (e.g., background fluorescence generated at the start of the amplification cycle) may provide more robust training than training based only on one or more data subsets associated with peak amplification (e.g., at least a subset containing the exponential phase), which in turn may enhance the ability of the trained machine learning system to accurately estimate an unknown quantity of the target organism.

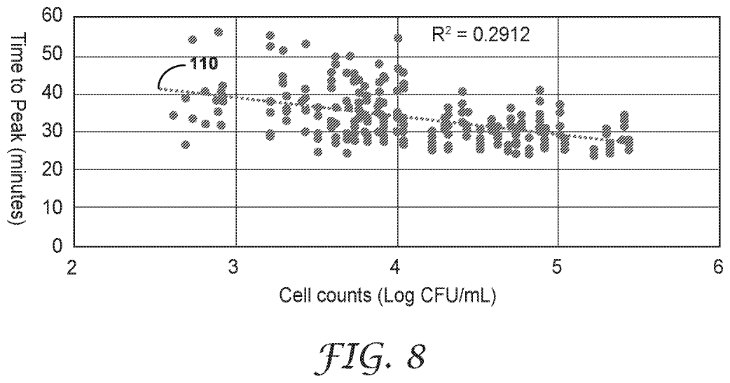

FIG. 8 illustrates time to peak amplitude versus cell count in a LAMP molecular assay of five Salmonella strains, in accordance with one aspect of this disclosure. In some example approaches, quantification of DNA-based assays is performed using high quality DNA and a single response value from a DNA amplification reporter. This response value, usually fluorescence or bioluminescence, may be based on the signal surpassing a preset threshold value or on a peak amplitude value. FIG. 8 illustrates a linear model with the response from five Salmonella strains where n=480. In some examples, it may be desirable to estimate an initial quantity of more than one strain or species (e.g., within a genus) of a target organism in a sample, as more than one of such strains or species may be pathogenic. An approach that uses multiple strains of a target organism will be discussed in the context of FIG. 8.

In the example shown in FIG. 8, culture preparation was performed by inoculating 10 mL of Buffered Peptone Water (BPW, 3M Company, St. Paul) with a single colony from an agar plate corresponding to each strain (Table 1). The inoculated broths were incubated at 37° C. for 18 h.

TABLE 1

| Strain | Reference¹ |
|---|---|
| Salmonella enterica subsp. enterica serovar Typhimurium | ATCC ® 14028 ™ |
| Salmonella enterica subsp. enterica serovar Enteritidis | ATCC ® 13076 ™ |
| Salmonella enterica subsp. enterica serovar Hadar | TC 164 |
| Salmonella enterica subsp. enterica serovar Infantis | ATCC ® 51741 ™ |
| Salmonella enterica subsp. enterica serovar Kentucky | TC 251 |

¹American Type Culture Collection and Tecra ™ Collection.

For enumeration, the cultures were serially diluted in Butterfields Buffer and plated onto 3M™ brand Petrifilm™ Aerobic Count (AC) Plates (3M Company) (hereinafter “Petrifilm AC plates”) following manufacturer's instructions. The cultures were kept at 4-8° C. until plate count results were obtained. The counts obtained were used to estimate the number of cells used for the detection using 3M™ brand Molecular Detection Assay 2—Salmonella (3M Company) (hereinafter “MDA2—Sal”). A final plate count was conducted using Petrifilm AC plates at the time of conducting the detection assay.

These final plate counts were used for reporting the concentration of cells.

In one example approach, each strain was serially diluted in Butterfield's Buffer to approximately 10², 10¹, 10⁴, 10⁵ and 10⁶ colony forming units (CFU) per milliliter. Aliquots from each dilution were analyzed using MDA2—Sal following manufacturer's instructions. MDS software supplied by 3M Company was then used to determine the time-to-peak, a response to the amplification of the target sequence. FIG. 8 illustrates the time-to-peak response of each aliquot at the cell concentration for the aliquot determined from the final plate count for each strain. A dataset of time-to-peak for known concentrations of cells was then used to train a Decision Forest Regression model and a Boosted Decision Tree model. Both approaches yielded coefficients of determination of approximately 0.75. The same dataset used to train a linear regression model around line 110 yielded a coefficient of determination R² of approximately 0.2912. Other regression techniques, such as support vector regression, random forest regression, ridge regression, logistic regression, Lasso, and nearest neighbor regression, may also be used to train models based on data sets of time-to-peak for known concentrations of cells.

Time-to-peak response is not always the best measure of cell count. Differing matrices (i.e., substances other than a pure culture in a sample or molecular components in food sample) may prevent good agreement between time-to-peak response and actual cell counts. A particular count of cells of a Salmonella strain may, for instance, produce different time-to-peak measurements depending on the matrix in which the cells are located. For example, different time-to-peak measurements may result from a particular count of cells of the Salmonella strain in a salmon matrix versus a shellfish matrix, or in other different matrices. In some example approaches, measurements of parameters such as light intensity over time across a nucleic acid amplification cycle provide a better representation of initial cell count. Even then, it may be advantageous to train a machine learning system with different matrices to more accurately estimate quantity of a target organism within a particular matrix.

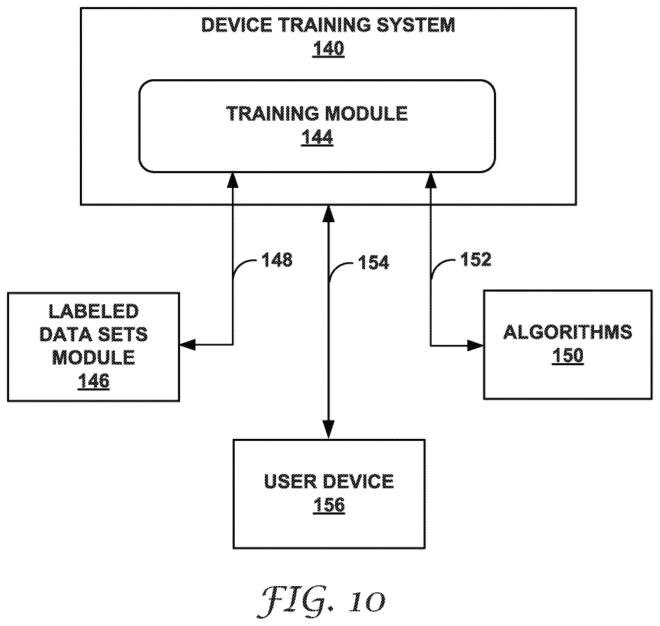

FIGS. 9A-9C and 10 illustrate example systems and techniques for training and using a machine learning system to quantify organisms in biological assay, such as biological assay that include the Salmonella species described with respect to FIG. 8. FIGS. 9A-9C are flow diagrams illustrating example approaches for training a machine learning system and for employing the trained machine learning system to quantitate a target organism of interest in a sample, in accordance with aspects of this disclosure. FIG. 10 is a block diagram illustrating a system that may be used in an example technique for training the machine learning system of FIGS. 9A and 9B. The systems and methods for training and using a machine learning system described below with respect to example techniques of FIGS. 9A-9C, improve the predictive power of the constructed model compared to models based on time-to-peak measurements such as shown in the model illustrated in FIG. 8. Moreover, in contrast to traditional methods, such systems and methods for training and using machine learning systems perform well when there is a particular matrix involved (e.g., the poultry rinse matrix) and not just a pure culture.

In the example approach of FIG. 9A, a nucleic acid amplification device 8 in system 6 is used to test assays having known cell concentrations of a target organism to obtain a data set for each assay (112). The assays may be from cultures, from matrices, or both. Each data set is then labeled with a quantity reflective of the quantity of target organisms detected in each respective array by the nucleic acid amplification device (114). System 6 then trains a machine learning system using the labeled data sets (116). In some example approaches, the method further includes estimating a quantity of the target organism in an assay using the trained machine learning system (118). In some example approaches, each data set is labeled with a quantity obtained from the respective assay using an alternative quantitation method such as, for example, MPN.

In some example approaches, each data set includes time-series measurement samples of the light intensity detected by detector 16 during an amplification cycle. Each data set is labeled with known cell concentration of its respective assay and the labeled data set is then used to train a machine learning system 25 or 35 as detailed below. Machine learning system 25 or 35 is then used to estimate a quantity of the target organism in each assay. In some example approaches, a different data set is used for each matrix or type of matrix. A matrix representing target organisms in cheese may be used, for example, to train a machine learning system 25 or 35 for use in quantitating target organisms in a cheese factory.

FIG. 9B is another flow diagram illustrating an example approach for obtaining a data set from a matrix having a known cell concentration and for using the data set to train a machine learning system to quantitate a target organism of interest in a matrix. In the example approach of FIG. 9B, the method includes obtaining a sample from a matrix to be tested (122), adding enrichment medium (124) to the sample, diluting the sample (126) and then incubating the sample (128) before analyzing the sample with a nucleic acid amplification device to produce a data set (130). The method further includes testing the sample using an alternate method (such as MPN) to produce a label for each data set with the known cell concentration of the sample that produced the data set (120). The data set and its associated label are then used to train the machine learning system (132).

In some example approaches, each data set includes light intensity measurements made over time during one or more amplification cycles. In some such example approaches, each data set includes the time-series measurements of light intensity captured across the whole of the amplification cycle. In some example approaches, such data sets also include measurements made during a period at the start of the amplification cycle where the data is typically either not captured, discarded or otherwise suppressed by nucleic acid amplification device 8. In some example approaches, each data set includes light intensity measurements made in a first period before Tₘₐₓ, light intensity measurements made in a second period of time including Tₘₐₓ, and light intensity measurements made in a third period of time occurring after Tₘₐₓ.

In the example approaches of FIGS. 9B and 9C, steps 120-132 may be carried out in an example technique for training the machine learning system while steps 122-130 and 136 may be carried out in an example technique for using the trained machine learning system. Although one or more aspects of the two workflow techniques may be described herein with respect to one or more specific nucleic acid amplification and detection components, in other examples, the techniques of FIGS. 9B and 9C may be performed using one or more other nucleic acid amplification and detection components.

In one such example approach of using a machine learning system to estimate a quantity of a target organism, the technique of FIG. 9C includes receiving a sample of a matrix (122), such as by a laboratory worker or automated equipment. The matrix may be, for example, a matrix in which the target organism may be found, such as the poultry rinse matrix described with respect to FIG. 8 or a portion of a raw material of a food product or end product of a food product. Upon receiving the matrix, the laboratory worker or equipment adds an appropriate enrichment medium configured to enable growth of the target organism within the sample containing the target organism and the matrix to a detectable limit (124). In some examples, such as examples in which a PCR technique is used for amplification of target nucleic acid, an appropriate enrichment medium may have a characteristic of being less likely to interfere with the fluorescence emitted during PCR than one or more otherwise appropriate enrichment media, such as by emitting less background fluorescence relative to other appropriate media. Next, in some example approaches, the worker or equipment prepares a 1:10 dilution of the resulting enrichment solution (126). As discussed below with respect to FIGS. 11 and 12, the use of a 1:10 dilution may increase the specificity of the trained machine learning system for the target organism. Any other suitable dilution may be used, such as 1:100 or 1:1000. The amount of dilution will, in some example approaches, depend on system characteristics such as the type of organism targeted and the particular amplification technique.

Next, the sample within the enrichment solution is incubated to allow enrichment of the target organism (128). In some examples, the sample may be incubated at about 35-42° C. for about 4-24 hours, or at any other suitable temperature and period of time that may enable suitable growth of the target organism. In other examples, an enrichment step may not be used, but instead the nucleic acid may be extracted from a sample without enrichment. Following incubation, if used, the sample is analyzed via, in some example approaches, amplification and detection of the target nucleic acid associated with the target organism (130). For example, the target nucleic acid may be amplified and detected using a nucleic acid amplification device 8 having a light detector 16 such as the MDS. The MDS, for example, may be configured to amplify the target nucleic acid by carrying out a LAMP technique and may then detect bioluminescence emitted by a light-emitting species within the sample (e.g., luciferin) using detector 16. By combining LAMP with bioluminescence detection, nucleic acid amplification devices such as the MDS may make molecular detection of foodborne pathogens simpler and faster, thereby providing users with speed and ease in simultaneously identifying one or more target organisms (e.g., one or more species or strains of Salmonella, Listeria, Listeria monocytogenes, E. coli O157 (including H7), Campylobacter, Cronobacter and/or other target organisms) in food and/or environmental samples. In other example approaches, the techniques of FIGS. 9A-9C are carried out using a different LAMP platform or using a PCR platform or a different nucleic acid amplification platform.

In some example approaches, the amplitude of light generated early in an amplification cycle (e.g., before phase 94 or phase 104) may be suppressed (e.g., not recorded) so as to not confuse users with background activity. It has been found, however, that such information may be helpful in training the machine learning system. Therefore, in one example approach, the data set includes time-series measurements made before phase 94 in FIG. 6. In a similar example approach, the data set includes time-series measurements made before phase 104 in FIG. 7.

In some example approaches, labeled data sets are produced by expert inspection of individual samples on which nucleic acid amplification has been performed. In one such example approach, an expert receives data sets associated with the samples, determines a quantity of organisms and/or target nucleic acid in the sample (via, for example, one of the traditional quantification techniques described above such as MPN) and labels each data set with the determined quantity value. The labeled data sets are then used to train a machine learning system, as depicted in FIGS. 9A and 9B.

In some example approaches, data sets include time-series measurements taken at predetermined intervals (e.g., 25 seconds) across the whole of the amplification cycle. In other example approaches, data sets include data selected from certain phases of the amplification cycle. For instance, a data set may include data from one or more of phases 94, 96 and 98 in FIG. 6 or from one or more of phases 104, 106 and 108 in FIG. 7. For example, where (130) includes a LAMP technique, the data set may include one or more data subsets as described with respect to FIG. 6. For example, the data set may include a first data subset representing time-series measurement samples of light emitted up to a first point in time in the amplification cycle, the first point in time occurring prior to a peak amplitude of the light emitted over the amplification cycle, a second data subset representing time-series measurement samples of light emitted after the first point in time but before a second point in time in the amplification cycle, the second point in time occurring after the peak amplitude, and a third data subset representing time-series measurement samples of light emitted after the second point in time in the amplification cycle. A computing device (e.g., processing circuitry 30 of external device 28 of FIG. 2 or any other suitable computing device) then trains a machine learning system to predict the initial concentration (i.e., quantity) of the target organism of interest (132). For example, the computing device may label a data set, and/or one or more subsets of the data set, with an estimate of the quantity of the target organism within the biological assay associated with the respective data set or data subset. The computing device then trains the machine learning system with the labeled data sets (or data subsets) and/or matrix identity to estimate a quantity of the target organism within the sample, resulting in a trained model. The computing device then may store the parameters of the trained machine learning system to one or more storage components of a system, such as a memory of a computing device, user device 20, a memory of a computing device of access point 24, and/or to any other suitable location.

In a workflow technique associated with using a trained machine learning system to calculate a quantity of the organism of interest, the technique of FIG. 9C includes carrying out steps 122-130 substantially as described above with respect to an example technique for training the machine learning system, although the matrix at (122) may be a sample of a raw food material, an end food product, or an environmental sample that may contain a target organism of interest instead of a known quantity of the target organism. In such examples, a nucleic acid amplification and detection system, such as the MDS or another system configured to carry out LAMP or PCR and detect light emitted by light-emitting species during one or more amplification cycles, may capture a data set, the data set comprising time-series measurement samples of the light emitted by the light-emitting species during the amplification cycle and analyze the data set (130). The data set is then analyzed based on the trained machine learning model to arrive at an estimate of the quantity of the target organism in the matrix (136).

In some such examples the data set may include one or more data subsets corresponding to one or more portions of an amplification cycle, such as in a manner similar to data subsets with which the machine learning system is trained. For example, a data set corresponding to a sample containing an unknown quantity of a target organism may include a first data subset representing time-series measurement samples of light emitted up to a first point in time in the amplification cycle, the first point in time occurring prior to a peak amplitude of the light emitted over the amplification cycle, a second data subset representing time-series measurement samples of light emitted after the first point in time but before a second point in time in the amplification cycle, the second point in time occurring after the peak amplitude, and a third data subset representing time-series measurement samples of light emitted after the second point in time in the amplification cycle. A computing device configured to receive the first, second, and third data subsets (e.g., computing device 42 of user device 20, a computing device of access point 24, or any other suitable computing device) applies the trained machine learning system to the data subsets (136) and calculates the concentration (e.g., quantity) of the target organism of interest in the sample. In some examples, the computing device then may store one or more such estimated quantities to one or more storage components of a system, such as a memory of an MDS, a memory of a computing device user device 20, a memory of a computing device of access point 24, and/or to any other suitable location.

In some example approaches, separate machine learning systems are trained as a function of the type of matrix being tested. For instance, a separate system may be trained for testing cheese, or for testing feed, with the parameters of each machine language machine learning system stored in memory based on the type of matrix being tested.

FIG. 10 is a block diagram illustrating a device training system, in accordance with one aspect of this disclosure. In the example shown in FIG. 10, device training system 140 includes a training module 144 connected to labeled data sets module 146 via link 148. Training module 144 is also connected to machine learning system storage 150 via link 152. In some example approaches, device training system 140 is connected via a link 154 to a user device 156. In one example approach, training module 144 includes a computing device, one or more storage components and a user interface. For example, device training system 140 may include a computing device of external device 28 and memory 32 of FIG. 2. In one example approach, training module 144 receives labeled data sets from labeled data sets module 146. In some such example approaches, each labeled data set includes a target organism quantity associated with a sample and measurements of light detected during an amplification cycle of the sample by a nucleic acid amplification device 8. Training module 144 trains a machine learning system with the labeled data sets and stores parameters associated with the machine learning system in algorithms 150.

It can be time consuming to obtain labeled data, as the production of labeled data requires inspection by an expert of individual samples or the generation of reference samples that can be compared to the samples being measured. In the alternative, in the absence of labeled data, one may approximate labeled data by carefully controlling the environment in which samples are taken. An example approach for generating labeled data from reference samples will be discussed next.

For enumeration, the cultures were serially diluted in Butterfields Buffer and plated onto 3M™ brand Petrifilm™ Aerobic Count (AC) Plates (3M Company) (hereinafter “Petrifilm AC plates”) following manufacturer's instructions. The cultures were kept at 4-8° C. until plate count results were obtained. The counts obtained were used to estimate the number of cells used for the detection using 3M™ brand Molecular Detection Assay 2—Salmonella (3M Company) (hereinafter “MDA2—Sal”). A final plate count was conducted using Petrifilm AC plates at the time of conducting the detection assay. These final plate counts were used for reporting the concentration of cells. In one example approach, each strain was serially diluted in Butterfield's Buffer to approximately 10², 10³, 10⁴, 10⁵ and 10⁶ CFU per milliliter. Aliquots from each dilution were analyzed using MDA2—Sal following manufacturer's instructions. MDS software supplied by 3M Company was then used to determine the time-to-peak, a response to the amplification of the target sequence.

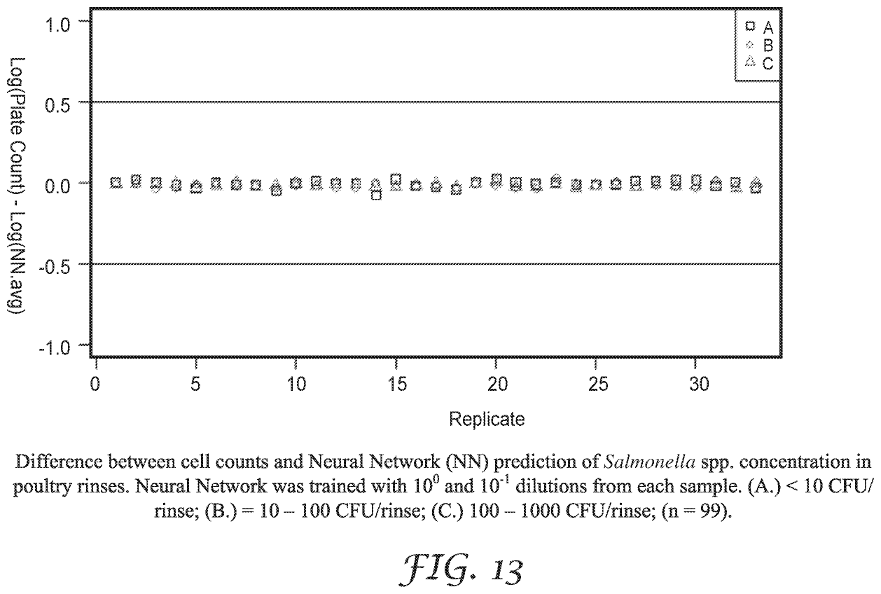

FIGS. 11-14 illustrate techniques for using trained machine learning systems to predict the quantity of five Salmonella species in sample poultry rinses. FIG. 11 illustrates a technique for training a machine learning model to estimate cell counts of target cells inoculated into a matrix and a technique for using the trained machine learning model to estimate cell counts in a matrix based on the trained model, in accordance with one aspect of this disclosure. In one example approach of the technique of FIG. 11, poultry rinses are prepared by adding 400 mL of BPW to a whole poultry carcass and mixing by hand (200). After removing the carcass, 10-mL aliquots of the rinses are inoculated with approximately 10¹, 10², 10³ and 10⁴ cells/sample of each of the strains in Table 1 above (202). The strains are prepared as described in the example used in the discussion of FIG. 8 above. In one example approach, an enrichment medium is added to each aliquot (204). In the approach shown in FIG. 12, the matrix is not diluted at 206 while in the example shown in FIG. 13, the enriched matrix is diluted in a 1:10 dilution (206). The inoculated rinses are then incubated at 41.5° C. for 7 hours (208). After the incubation, aliquots from the rinses are analyzed using MDA2—Sal following manufacturer's instructions. A signal response (relative light units) for each aliquot is captured as a series of measurements taken over approximately 60 min (i.e., over the DNA amplification cycle of the MDS) and data representing the measurements is stored in a data set associated with each aliquot (210).

In some example approaches, each data set includes a first, second and third subset of data. The first subset of data includes measurements captured before a first point in time in the amplification cycle, the first point in time occurring prior to a time Tmax, where the time Tmax corresponds to a time to a peak amplitude of the parameter being measured in the nucleic acid amplification cycle. The second data subset includes measurements captured after the first point in time but before a second point in time in the nucleic acid amplification cycle, the second point in time occurring after Tmax. The third data subset includes measurements captured after the second point in time in the nucleic acid amplification cycle.

In training mode, each data set is labeled with a cell concentration based on an estimate of the initial cell concentration in the aliquot associated with the data set. In other example approaches, each data set is labeled with a value obtained via another method, such as MPN. The labeled data sets are then used to train a machine learning model such as a Neural Network to estimate cell concentrations in matrices (212).

In production mode, the machine learning system receives a data set for each matrix analyzed by the nucleic acid detector and determines an initial concentration of a target organism in the matrix by applying the data set to the trained machine learning model (214). An example showing the differences between the predicted cell concentrations from Neural Network-based machine learning model and the cell concentrations determined from corresponding plate counts are shown in FIG. 12. In this example shown in FIG. 12, the model is able to explain 84% of the overall variability in the dataset.

In one example approach, the techniques of FIG. 11 are carried out at each known-level inoculation of the organism of interest. In one such example approach, the process is repeated a sufficient number of times at each of multiple levels of CFU inoculations to establish a representative sample of data sets. In some such example approaches, this may require running 100 or more amplification cycles at each inoculation level for each type of matrix. In one such example approach, the levels include a level of below 10 CFUs, such as 1-10 CFUs, a level between 10-100 CFUs, a level between 10-1000 CFUs, a level above 1000 CFUs, and/or any other suitable known inoculation level. For each known-level inoculation, the nucleic acid amplification and detection device may capture data sets comprising time-series measurement samples of the light emitted by the light-emitting species during each amplification cycle.

FIG. 13 illustrates log differences between cell count predictions made by a trained machine learning system and different cell counts of Salmonella cells inoculated into a poultry rinse matrix and also into a 1:10 dilution of the poultry rinse matrix, in accordance with one aspect of this disclosure. An approach similar to the approach used in the example of FIG. 12 may be used. However, in the example of FIG. 13, a 1:10 dilution of the rinse (206 of FIG. 11) was also incubated and incorporated into the analysis.

In one such example approach, poultry rinses were prepared by adding 400 mL of BPW to a whole poultry carcass and mixing by hand. After removing the carcass, 10-mL aliquots of the rinses were inoculated with approximately 10¹, 10², and 10³ cells/sample of each of the strains in Table 1 above. The strains were prepared as described in the example used in the discussion of FIG. 8 above. For each rinse, a 1:10 dilution was also prepared in BPW. The inoculated rinses and the dilutions were incubated at 41.5° C. for 7 hours. After the incubation, aliquots from the all the samples were analyzed using MDA2—Sal following manufacturer's instructions. As in the example discussed for FIGS. 11 and 12 above, the entire signal response (relative light units) over time (60 min), during the DNA amplification, was extracted, labeled and used to train a Neural Network algorithm. In one such example approach, the response data from both the 10⁰ and 10¹ dilutions were treated as a single data point and labeled with the cell concentration that was inoculated into the rinse. The differences between the predicted cell concentrations from the Neural Network model and the cell concentrations from corresponding plate counts are shown for this example approach in FIG. 13.

In this case, the model was able to explain 99% of the overall variability in the dataset, a significant improvement over the linear model shown in FIG. 8 and also an improvement over the example approach of FIG. 12. The result illustrated in FIG. 13 indicates that in some examples it may be desirable to include such a dilution in carrying out techniques for training a machine learning system.

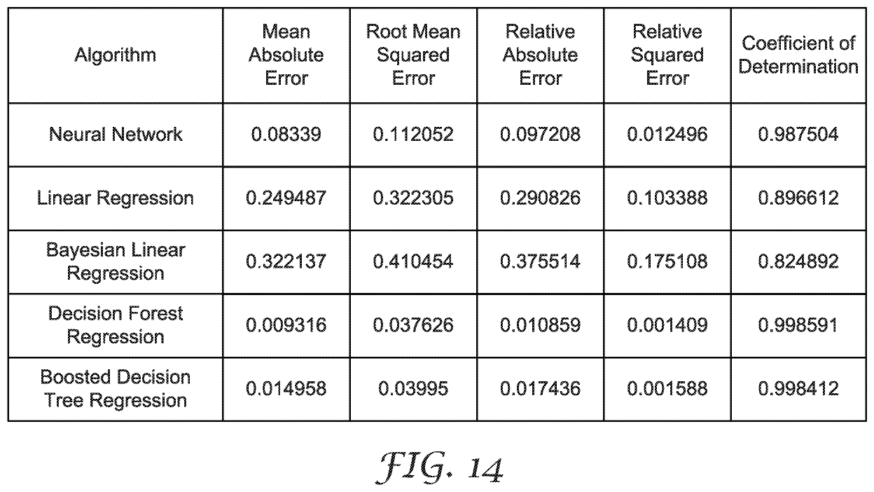

FIG. 14 illustrates various metrics for measuring performance for regression used for cell count prediction using a variety of machine learning techniques, in accordance with aspects of this disclosure. In the example shown in FIG. 14, a poultry rinse was prepared and tested using the method described above in the example approach of FIG. 13. As in the example shown in FIG. 13, the response data from both the 10⁰ and 10¹ dilutions were treated as a single data point and labeled with the cell concentration that was inoculated into the rinse. The labeled data sets were then used to train a neural network model, a linear regression model, a Bayesian linear regression model, a Decision Forest regression model, and a Boosted Decision Tree regression model. Each of the models were used to predict cell concentrations. FIG. 14 provides metrics comparing the results from each machine learning model as compared to traditional plate counts.

Thus, as described herein, it may be advantageous to apply a trained machine learning system to data sets derived from nucleic acid amplification biological assay of a nucleic acid associated with one or more target organisms. Compared to linear models based on standard curves such as the model shown in FIG. 8, training and using a machine learning system, such as described below with respect to FIGS. 9A-C and 11, improves the predictive power of the constructed model. For example, by applying a trained machine leaning machine learning system to the dataset of FIG. 8, such a method resulted in an R² of 0.75 using either a Decision Forest Regression or a Boosted Decision Tree. Thus, FIGS. 12 and 13 illustrate that in addition to reducing or eliminating the need to isolate pure DNA for pathogen quantification, the systems and methods described herein may perform well for multiple strains or species of an organism of interest, such as multiple Salmonella species.

As noted above, assays based on molecular methods such as nucleic acid amplification ((e.g., LAMP or PCR) may be affected by the presence of matrix-derived substances which can interfere or prevent the reaction from performing correctly. In food production, matrix-derived substances, such as spices and environmental samples, may act as inhibitors that can interfere with nucleotide amplification assays such as PCR and LAMP, leading to false negative results or to positive detection with incorrect quantification.

It can be difficult to eliminate inhibition or to limit its effects. Careful sample treatment may be used, for instance, to remove inhibitory substances. No sample treatment, however, can be relied on to completely remove inhibitory substances. Inhibition may be detected via amplification controls; such controls may be used, for instance, to verify that the assay has performed correctly. Amplification controls adds expense and complexity to molecular methods.

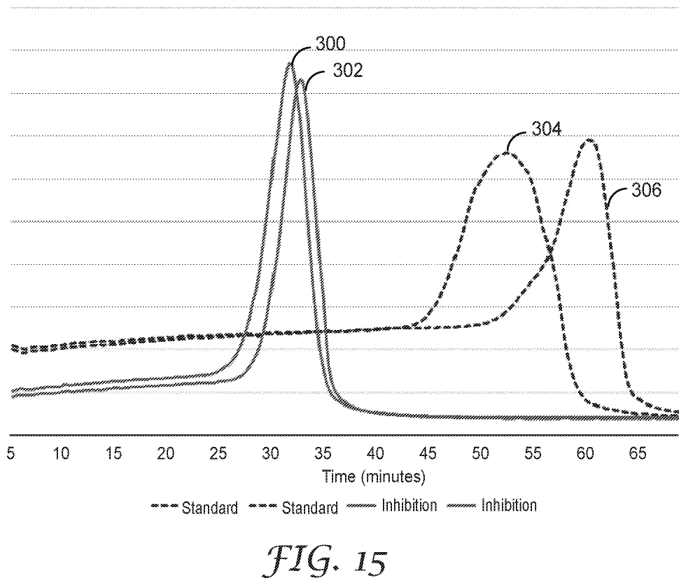

FIG. 15 is a conceptual drawing illustrating nucleic acid amplification in standard and inhibited samples during a LAMP amplification cycle, in accordance with one aspect of this disclosure. As noted above, in LAMP, the emission of bioluminescence may be detected by a detector of a nucleic acid amplification device configured for LAMP, such as detector 16 of nucleic acid amplification device 8 of FIGS. 1 and 2. Data representing time-series measurements of the intensity of the bioluminescence are stored as a data set. In some examples, the mechanism for generating light during a LAMP technique illustrated in FIG. 15 may provide one or more other benefits, such as enabling real-time detection of nucleic acid amplification occurring during the LAMP amplification cycle over a relatively short period of time, such as about 15 minutes.

Inhibition can be exhibited in several ways. Time-to-peak is one characteristic to look at when assessing inhibition or other issue in the reaction (poor reaction performance due to primer design). In FIG. 15, the samples illustrate a “normal” run (300, 302) and the late peaks of runs (304, 306) with a matrix known to cause inhibition. Inhibited samples may tend to exhibit a longer time-to-peak RLU emission and a lower maximum amplitude. Similarly, in PCR, the presence of inhibitors may prevent the polymerase from extending the DNA in the time allowed, which may result in incomplete amplification products and may prevent the detection of the target organism.

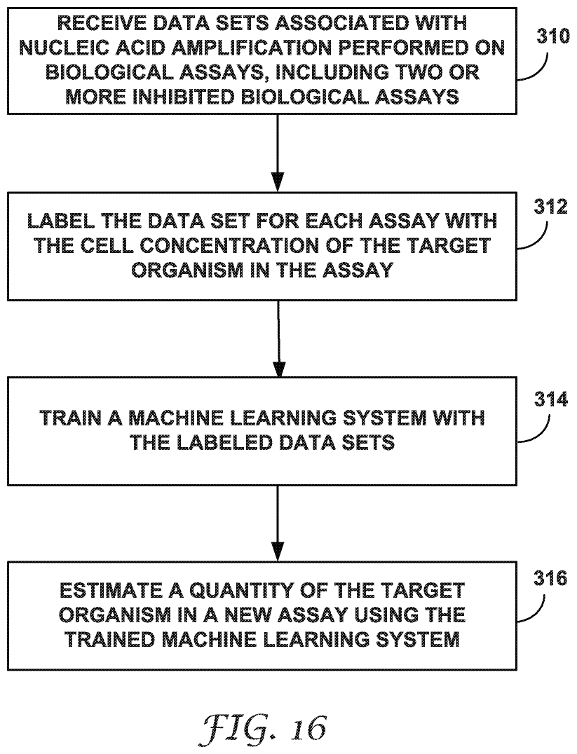

The difference in time to peak may also, however, be the response to different DNA concentration. It can be difficult, therefore, to determine whether the shift of the peak is a product of DNA concentration or due to some kind of inhibition. The approach described below in the context of FIG. 16 recognizes and corrects for quantification due to inhibition by training a machine learning system with data sets from assays with different levels of inhibition.

FIG. 16 is a flow diagram illustrating an example technique for training a machine learning system to quantify target organisms in inhibited samples, in accordance with one aspect of this disclosure. This approach can be used, for instance, to quantify organisms in biological assay, such as biological assays that include the Salmonella species described with respect to FIG. 8. Systems and methods based on this approach improve the predictive power of the constructed model compared to models based on time-to-peak measurements such as shown in the model illustrated in FIG. 8. Moreover, in contrast to traditional methods, such systems and methods for training and using machine learning systems perform well when there is a particular matrix involved (e.g., the poultry rinse matrix) and not just a pure culture, even in the face of inhibitory substances.

In the example shown in FIG. 16, a machine learning system (such as machine learning systems 25 and 35 of FIGS. 1 and 2, respectively) is trained to quantify a target organism present in a biological assay. In one example approach, a device training system 140 such as shown in FIG. 10 receives a large number of data sets, each data set associated with a biological assay being tested for a target organism (310). A significant number of the biological assays include inhibitory substances.

In one example approach, each data set includes data collected by a detector across one or more nucleic acid amplification cycles. The data includes activity measurements taken at different times during the one or more nucleic acid amplification cycles and represents nucleic acid amplification of a target nucleic acid associated with the target organism within the biological assay. In some example approaches, the activity measurements include time-series measurements of relative light units (RLU) emitted by a light-emitting species (e.g., luciferin) in the biological assay containing the target nucleic acid. As noted above, exponential amplification of the target nucleic acid during a LAMP amplification cycle produces a bioluminescence signal having both a rapid increase in RLU and a rapid decrease in RLU. In such examples, the curve traced by measurements of RLU emission corresponds to the quantity of the target organism present in the assay, even in the face of inhibition. Thus, parameters representing the curve traced during the one or more amplification cycles may be used by device training system 140 to train a machine learning system to estimate a quantity of a target organism in a sample. The relevant parameters may include time-to-peak but, as noted above, time-to-peak response is not always the best measure of cell count. Measurements of parameters such as light intensity over time across a nucleic acid amplification cycle provide a better representation of initial cell count. In some example approaches, the measurement of light intensity over time includes intensity measurements made during the amplification cycle but before the amplification of the target nucleic acid is detected. Even then, it may be advantageous to train a machine learning system with different matrices and different levels of inhibition to more accurately estimate quantity of a target organism within a particular matrix.

In some examples, the data set used to train a machine learning system (such as, for example, a neural network) includes data captured as a set of time-series measurement samples of bioluminescence captured across the entirety of the amplification cycle for both standard and inhibited biological assays. In one such LAMP example, luminescence measurements are taken approximately every 5 seconds, which may be accumulated as measurements at 10, 15, 20, and/or 25 second intervals across the amplification cycle for reporting purposes.

Returning to the discussion of FIG. 16, each data set received by device training system 140 is labeled with an estimate of the quantity of the target organism present within the associated biological assay (312). The labeled data sets are then used to train a machine learning system to estimate a quantity of the target organism within a selected biological assay (314). In one example approach, machine learning system the training based on the activity measurements stored in each of the plurality of data sets and an estimate of the quantity of the target organism present in the biological assay associated with each respective data set. In one example approach, the labeled data sets are used to train models such as a neural network model, a linear regression model, a Bayesian linear regression model, a Decision Forest regression model, and a Boosted Decision Tree regression model, as discussed above in the context of FIG. 14.

In the example approach of FIG. 16, a nucleic acid amplification device 8 in system 6 is used to test assays, including inhibited assays, having known cell concentrations of a target organism to obtain a data set for each assay. The assays may be from cultures, from matrices, or both. Each data set is then labeled with a quantity reflective of the quantity of target organisms detected in each respective array by the nucleic acid amplification device (312). System 140 then trains a machine learning system using the labeled data sets (314). In some example approaches, the method further includes estimating a quantity of the target organism in an assay using the trained machine learning system (316). In some example approaches, each data set is labeled with a quantity obtained from the respective assay using an alternative quantitation method such as, for example, MPN. In some example approaches, a different data set is used for each matrix or type of matrix. A matrix representing target organisms in cheese may be used, for example, to train a machine learning system 25 or 35 for use in quantitating target organisms in a cheese factory.

In one example approach, a system for quantifying a target organism present in a sample includes a detection device (such as nucleic acid amplification device 8 in FIGS. 1 and 2) configured to amplify and detect a target nucleic acid associated with the target organism and a machine learning system (such as machine learning system 25 in FIG. 1 or machine learning system 35 in FIG. 2) configured to receive the activity measurements and to estimate the quantity of the target organism in the sample based on the activity measurements. The detection device includes a detector and a reaction chamber configured to receive an assay of the sample and to amplify the target nucleic acid in the assay over a nucleic acid amplification cycle. The detector is configured to capture, at different times within the nucleic acid amplification cycle, activity measurements representative of the quantity of the target nucleic acid present in the assay.

In one such example approach, the machine learning system is trained with a plurality of training data sets, each training data set associated with a training assay and including activity measurements representative of the quantity of the target nucleic acid present in the training assay, wherein the training is based on the activity measurements stored in each training data set and an estimate of the quantity of the target organism present in the training assay associated with each respective training data set. The training assays include assays with different levels of inhibition.

It is becoming increasingly important to quantitate pathogens as part of food, feed and water production safety. For instance, for certain pathogens, such as B. cereus, S. aureus, and Vibrio species, producers may be required to go beyond merely detecting the presence or absence of the pathogen and, instead, may be required to provide quantitative information on the pathogen. Furthermore, regulations in certain countries may require quantitative information for risk assessments; mere presence/absence criteria may not be adequate to provide the needed information. For example, in Europe, the maximum allowable level of L. monocytogenes in certain products varies depending on the product's intended use.

Even where not required by regulations, methods for obtaining quantitative pathogen information on pathogens may be used to develop more effective intervention processes and/or more effective processes for monitoring pathogen levels than can be achieved using presence/absence criteria. Food, feed and water producers may, for instance, be able to use such methods to evaluate the effectiveness of current intervention procedures in reducing pathogen levels in their products. The ability to determine not only the presence of, but also the quantity of, microorganisms present in a biological assay is, therefore, becoming increasingly critical not only in quantifying the pathogen but also in assessing the efficacy of steps taken to control pathogens in food, feed, water and corresponding processing environments. The ability to determine the quantity of a target organism in the presence of inhibitors is especially important. The techniques described above provide fast, accurate, quantitation of pathogens in a sample and may eliminate the need for amplification controls. Furthermore, since each type of microorganism is associated with one or more nucleic acids, the techniques described above can be used to determine cell concentrations in samples containing any type of microorganism.

Various examples have been described. These and other examples are within the scope of the following claims.

1. A system for quantifying a target organism present in a sample, comprising:
a detection device configured to amplify and detect a target nucleic acid associated with the target organism, the detection device comprising:
a reaction chamber configured to receive an assay of the sample and to amplify the target nucleic acid in the assay over a nucleic acid amplification cycle; and
a detector, the detector configured to capture, at different times within the nucleic acid amplification cycle, activity measurements representative of the quantity of the target nucleic acid present in the assay and to store the activity measurements in a data set, wherein the data set includes:
a first data subset, the first data subset including the measurements taken prior to a time Tₘₐₓ, wherein the time Tₘₐₓ corresponds to a time in the nucleic acid amplification cycle when the measurements reach a maximum amplitude;
a second data subset, the second data subset including the measurements taken after the first point in time but before a second point in time in the nucleic acid amplification cycle, the second point in time occurring after Tₘₐₓ; and
a third data subset, the third data subset including the measurements taken after the second point in time in the nucleic acid amplification cycle; and
a machine learning system configured to receive the first, second, and third data subsets and to quantify the target organism in the sample based on the data subsets, wherein the machine learning system is trained to estimate a quantity of the target organism present in the assay based on the measurements present in the first, second, and third data subsets.

2-3. (canceled)

4. The system of claim 1, wherein the reaction chamber is configured to perform an amplification technique comprising one or more of LAMP, PCR, nucleic acid sequence-based amplification, or transcription-mediated amplification.

5-6. (canceled)

7. The system of claim 1, wherein the target organisms are microorganisms of one or more Salmonella species, one or more Listeria species, one or more Campylobacter species, one or more Cronobacter species, one or more E. coli strains, one or more Vibrio species, one or more Shigella species, one or more Legionella species, one or more B. cereus strains, or one or more S. aureus strains, one or more types of viruses, or one or more genetically modified organisms.

8. The system of claim 1, wherein the reaction chamber is further configured to amplify the target nucleic acid in the sample over a plurality of nucleic acid amplification cycles, and
wherein the detector is further configured to capture the measurements across the plurality of nucleic acid amplification cycles.

9. The system of claim 1, wherein the machine learning system is based on a regression model.

10. The system of claim 1, where the reaction chamber is further configured to receive a module, wherein the module includes:
a first plurality of reaction vessels, each vessel of the first plurality of reaction vessels containing a quantity of a lysis buffer solution; and
a second plurality of reaction vessels, each vessel of the second plurality of reaction vessels containing quantities of one or more reagents configured for use in a nucleic acid amplification reaction.

11. A method of making a system of claim 1, comprising:
receiving a plurality of data sets, wherein each data set is associated with a biological assay, each data set including measurements, performed on the associated biological assay by a nucleic acid amplification device of a specified type and collected over at least a portion of a nucleic acid amplification cycle, of a target nucleic acid detected within the associated biological assay, wherein the target nucleic acid is associated with a target organism;
labeling each data set with an estimate of the quantity of the target organism present within the associated biological assay; and
training a machine learning system with the labeled data sets to estimate a quantity of the target organism within a biological assay based on tests performed on the target nucleic acid in the biological assay by nucleic acid amplification devices of the specified type.

12. The method of claim 11, wherein the measurements are time-series measurements of light intensity collected over at least a portion of the nucleic acid amplification cycle wherein each data set includes:
a first data subset, the first data subset including the measurements taken prior to a time Tₘₐₓ, wherein the time Tₘₐₓ corresponds to a time in the nucleic acid amplification cycle when the measurements reach a maximum amplitude;
a second data subset, the second data subset including the measurements taken after the first point in time but before a second point in time in the nucleic acid amplification cycle, the second point in time occurring after Tₘₐₓ; and
a third data subset, the third data subset including the measurements taken after the second point in time in the nucleic acid amplification cycle.

13-15. (canceled)

16. The method of claim 11, wherein the nucleic acid amplification device performs an amplification technique comprising one or more of LAMP, PCR, nicking enzyme amplification reaction (NEAR), helicase-dependent amplification (HDA), nucleic acid sequence-based amplification (NASBA), or transcription-mediated amplification (TMA).

17. The method of claim 11, wherein the biological assays are from a matrix inoculated with two or more levels of organisms and wherein labeling each data set with an estimate of the quantity of the target organism includes setting the quantity as a function of the level of inoculation.

18. The method of claim 11, wherein the biological assays are from a plurality of matrix types and wherein training a machine learning system includes training the machine learning model to distinguish between matrix types.

19. A non-transitory computer-readable medium storing instructions that, when executed by processing circuitry, cause processing circuitry of a system of claim 1 to:
receive a data set generated by amplifying a quantity of a nucleic acid in the sample over a nucleic acid amplification cycle, wherein the nucleic acid is associated with the target organism, the data set including measurements, collected during the nucleic acid amplification cycle, that are representative of the quantity of nucleic acid in the sample, wherein the data set includes:
a first data subset, the first data subset including the measurements taken prior to a time Tₘₐₓ, wherein the time Tₘₐₓ corresponds to a time in the nucleic acid amplification cycle when the measurements reach a maximum amplitude;
a second data subset, the second data subset including the measurements taken after the first point in time but before a second point in time in the nucleic acid amplification cycle, the second point in time occurring after Tₘₐₓ; and
a third data subset, the third data subset including the measurements taken after the second point in time in the nucleic acid amplification cycle; and
apply a machine learning system to the data subsets, wherein the machine learning system is trained to estimate a quantity of the target organism present in the sample based on the measurements present in the first, second, and third data subsets.

20. The computer-readable medium of claim 19, wherein the measurements are time-series measurements of light intensity collected over the nucleic acid amplification cycle.

21. A system for quantifying a target organism present in a sample, comprising:
a detection device configured to amplify and detect a target nucleic acid associated with the target organism, the detection device comprising:
a reaction chamber configured to receive an assay of the sample and to amplify the target nucleic acid in the assay over a nucleic acid amplification cycle; and
a detector, the detector configured to capture, at different times within the nucleic acid amplification cycle, activity measurements representative of the quantity of the target nucleic acid present in the assay; and
a machine learning system configured to receive the activity measurements and to estimate the quantity of the target organism in the sample based on the activity measurements, the machine learning system trained with a plurality of training data sets, each training data set associated with a training assay and including activity measurements representative of the quantity of the target nucleic acid present in the training assay,
wherein the training is based on the activity measurements stored in each training data set and an estimate of the quantity of the target organism present in the training assay associated with each respective training data set, and
wherein the training assays include assays with different levels of inhibition.

22. The system of claim 21, wherein the activity measurements are time-series measurements of light intensity collected over at least a portion of the nucleic acid amplification cycle.

23. The method of claim 21, wherein the activity measurements are time-series measurements of light intensity collected over the nucleic acid amplification cycle.

24. A method of training a machine learning system of claim 21 to quantify a target organism present in a biological assay, the method comprising:
receiving a plurality of data sets, each data set associated with a biological assay, each data set including data collected by a detector during nucleic acid amplification of a target nucleic acid within the associated biological assay across one or more nucleic acid amplification cycles, wherein the data collected by the detector includes activity measurements taken at different times during the one or more nucleic acid amplification cycles, wherein the target nucleic acid is associated with the target organism and wherein the biological assays include biological assays with different levels of inhibition;
labeling each data set with an estimate of the quantity of the target organism present within the associated biological assay; and
training a machine learning system to estimate a quantity of the target organism within a selected biological assay, the training based on the activity measurements stored in each of the plurality of data sets and an estimate of the quantity of the target organism present in the biological assay associated with each respective data set.

25. The method of claim 24, wherein the activity measurements are time-series measurements of light intensity collected over at least a portion of one or more of the nucleic acid amplification cycles.

26. The method of claim 24, wherein the activity measurements are time-series measurements of light intensity collected over one or more of the nucleic acid amplification cycles.

27-30. (canceled)

31. A non-transitory computer-readable medium storing instructions that, when executed by processing circuitry, cause the processing circuitry to:
receive a plurality of data sets, each data set associated with a biological assay, each data set including data collected by a detector during nucleic acid amplification of a target nucleic acid within the associated biological assay across one or more nucleic acid amplification cycles, wherein the data includes activity measurements taken at different times during the one or more nucleic acid amplification cycles, wherein the target nucleic acid is associated with a target organism, and wherein the biological assays include biological assays with different levels of inhibition; and
train a machine learning system to estimate a quantity of the target organism within a selected biological assay, the training based on the activity measurements stored in each data set and an estimate of the quantity of the target organism present in the biological assay associated with each respective data set.
